(19) United States
(12) Patent Application Publication
(10) Pub. No.: US 2002/0013560 A1
(43) Pub. Date: Jan. 31, 2002

(54) Absorbent structure with integral vapor transmissive moisture barrier

(75) Inventors: John P. Erspamer, Lakeland, TN (US); Brian E. Boehmer, Bartlett, TN (US); John Perry Baker, Memphis, TN (US); David W. Wu, Bartlett, TN (US)

Correspondence Address: DARBY & DARBY, 805 THIRD AVENUE, 27TH FLR., NEW YORK, NY, 10022, US

(73) Assignee: BKI HOLDING CORPORATION

(21) Appl. No.: 09/854,179

(22) Filed: May 11, 2001

Related U.S. Application Data
(60) Provisional application No. 60/204,418, filed on May 12, 2000.
(60) Provisional application No. 60/252,544, filed on Nov. 22, 2000.

(51) Int. Cl. A61F 13/15; A61F 13/20

(52) U.S. Cl. 604/381; 604/368; 604/369

(57) ABSTRACT

Disclosed is a unitary absorbent core having a basis weight of about 75 grams per square meter or greater, including a fibrous absorbent layer having an upper fluid receiving surface and a lower surface with a hydrophobic vapor-transmissive moisture barrier integral with the lower surface of the absorbent layer. Also disclosed is a process for the production of the unitary absorbent core, including the steps of (a) producing a fibrous absorbent layer having upper and lower surfaces, and (b) applying to the lower surface of the fibrous absorbent layer a hydrophobic material which at least partially coats at least some of the fibers of the lower surface of the absorbent layer.

### RELATED APPLICATION DATA

This application claims priority under 35 U.S.C. 119 from U.S. provisional application serial numbers 60/204,418, filed May 12, 2000 and 60/252,544, filed Nov. 22, 2000, both of which are hereby incorporated by reference in their entirety.

### FIELD OF THE INVENTION

The present invention relates to absorbent structures useful in absorbent products such as disposable diapers, feminine hygiene products such as sanitary napkins and pantiliners, absorbent surgical pads, adult incontinence products, and other personal hygiene articles. More particularly, the present invention is directed to an absorbent structure including an absorbent core for absorbing and retaining fluids and a vapor-transmissive, moisture barrier integral therewith.

### BACKGROUND OF THE INVENTION

Feminine hygiene products, such as sanitary napkins, pantiliners, and other personal hygiene articles, are typically constructed with a body side liquid pervious topsheet, a liquid impervious backsheet and an absorbent structure, or core, sandwiched between the two. The construction of a typical product is such that the topsheet and backsheet are in intimate contact with the absorbent core and stabilized with an adhesive to keep them in intimate contact.

The backsheet is positioned on the garment-facing side of the product. The backsheet is necessary to provide a fluid barrier between the absorbent core and the user, preventing body exudates, imbibed by the absorbent core, from soiling the skin or clothing of the user.

The backsheet is typically impermeable to moisture vapor, that is, it has little or no vapor transmission properties. Thus, any vapors generated in use, such as perspiration or vaporization of volatiles by body heat, cannot escape and can cause skin wetness and discomfort while the product is being used.

There has been a trend in the state of the art to design “breathability” into absorbent products to improve skin health and comfort of the user. In such products, the liquid impervious backsheet is replaced with a microporous material that has vapor transmission properties. The backsheet barrier is interrupted with small pores to allow vapors to escape; thus, the backsheet is not continuous. However, there is also an opportunity for the fluid to strike through the backsheet material, particularly upon the application of pressure commonly encountered during normal use of the absorbent product, resulting in wetting of the skin or clothing of the user.

Accordingly, the use of a breathable, microporous backsheet in an absorbent product requires that additional steps be taken to protect the user from exposure to the body exudates imbibed by the absorbent core. One option is to overdesign the absorbent core such that it has sufficient absorbent capacity to hold the fluid and prevent it from exiting the core and striking through the backsheet. This results in thicker, less comfortable products and adds undesirable cost to the absorbent core.

Alternatively, an additional barrier material may be positioned between the absorbent core and the microporous backsheet. The additional barrier material may be a synthetic nonwoven or an apertured film. The material serves to provide additional barrier properties but also provides a space or gap between the absorbent core and the backsheet reducing the possibility that the fluid will strike through the core. The requirement for two separate layers adds expense and additional manufacturing steps to the structure.

Illustrative examples of absorbent products incorporating breathable backsheets are found in U.S. Pat. Nos. 3,932,682 to Loft et al., 3,989,867 to Sisson, 4,196,245 to Kitson et al., 4,306,559 to Nishizawa et al.,4,341,216 to Obenour, 4,609,584 to Cutler et al., 4,626,252 to Nishizawa et al., 4,681,793 to Linman et al., 4,713,068 to Wang et al., 4,713,069 to Wang et al., 4,758,239 to Yeo et al., 4,818,600 to Braun et al., 4,828,556 to Braun et al., 5,364,381 to Soga et al., 5,498,463 to McDowall et al., 5,560,974 to Langley and 5,843,056 to Good et al., all of which are hereby incorporated by reference.

Illustrative examples of absorbent products incorporating foams in absorbent products are found in U.S. Pat. Nos. 4,554,297 to Dabi, 4,740,528 to Garvey et al., 5,260,345 to DesMarais et al., 6,040,494 to Kalentun et al. and 6,107,356 to DesMarais, WO 99/61518 to Chen et al. and WO 00/13637 to Carlucci et al, all of which are hereby incorporated by reference.

The disclosure WO 00/13637 describes an absorbent article containing a single foam layer, characterized by an absorbent-core portion of the foam treated to be hydrophilic and a backsheet portion treated to be hydrophobic.

### SUMMARY OF THE INVENTION

It would be desirable to provide an absorbent core for use in an absorbent product having an integral vapor-transmissive moisture barrier. Such a core would be less expensive and easier to manufacture than prior art arrangements involving separately formed materials which must be combined and adhered together to form a product.

It is one object of the present invention to provide a unitary absorbent core, including a fibrous absorbent layer and a vapor-transmissive moisture barrier integral with one surface of the absorbent layer, which is thinner and more comfortable in use in disposable absorbent products, such as feminine hygiene products, diapers and adult incontinence products.

It is another object of the present invention to provide a unitary absorbent core including an integral vapor-transmissive moisture barrier which is less expensive to manufacture compared to absorbent cores incorporating apertured films, synthetic nonwovens and adhesives.

It is yet another object of the present invention to provide a unitary absorbent core including an integral vapor-transmissive moisture barrier which allows for simple conversion into a finished absorbent product, based on a reduction in the number of raw materials and process steps required to carry out the conversion.

It is another object of the present invention to provide a unitary absorbent core including an integral vapor-transmissive moisture barrier that is highly breathable, but also maintains a significant moisture barrier.

Another object of the present invention is to provide a unitary absorbent core including an integral vapor-transmissive moisture barrier and which also provides softness, drape and hand comparable to or better than that provided by a unitary absorbent core having an apertured film moisture barrier.

These and other objects are met by the present invention which is directed to a unitary absorbent core having a basis weight of about 75 gsm or greater comprising a fibrous absorbent layer having an upper fluid receiving surface and a lower surface with a hydrophobic vapor-transmissive moisture barrier integral with the lower surface of the absorbent layer. In a preferred embodiment, the barrier may be a hydrophobic latex emulsion applied to one surface of the absorbent layer. The absorbent core exhibits both a high water vapor transmission rate and a significant hydrostatic head (hydrohead) pressure. The absorbent core may have a moisture barrier which has a structure which substantially includes fibers coated with hydrophobic material, or it may have a moisture barrier which has a reticulated remnant of a barrier material emulsion extending from the lower surface region of the absorbent layer to form an outer reticulated foam barrier. A reticulated foam barrier is a very open structure, more open than the open celled structures known in the foam making art. Barriers of this type generally present a greater challenge to fluids trying to pass than barriers where the structure substantially includes fibers coated with hydrophobic material.

Within the scope of this invention is a process for the production of a unitary absorbent core having a basis weight of about 75 gsm or greater comprising a fibrous absorbent layer having an upper fluid receiving surface and a lower surface with a hydrophobic vapor- transmissive moisture barrier integral with the lower surface of the absorbent layer comprising:

(a) producing a fibrous absorbent layer having upper and lower surfaces,

(b) applying to the lower surface of the fibrous absorbent layer a hydrophobic material which at least partially coats the fibers of the lower surface of the absorbent layer. Desirably, the hydrophobic material is an emulsion polymer, which is applied in the form of a foam to a fibrous absorbent layer comprising synthetic and/or natural fibers in a nonwoven produced by an airlaid process. This aspect of this invention includes a unitary absorbent core produced by the process.

Further, this invention provides an absorbent article comprising:

(A) a liquid pervious top sheet, and

(B) a unitary absorbent core of this invention, which may also have

(C) a microporous backsheet.

The article may be in the form of an infant disposable diaper, a training pant, an absorbent surgical pad, an adult incontinence device, a sanitary napkin, a pantiliner or a feminine hygiene pad.

In a further aspect, this invention is a breathable nonwoven fibrous material having a basis weight of about 75 gsm or greater, a barrier effectiveness value of 30 mm or greater, and having a surface with a hydrophobic vapor-transmissive moisture barrier integral therewith comprising natural fibers, synthetic fibers or a mixture thereof, and a hydrophobic material which at least partially coats the fibers of a surface of the material.

In a further aspect, this invention includes a breathable, partially fibrous or nonfibrous nonwoven material or structure having a basis weight of about 45 gsm or greater, a barrier effectiveness value of 30 mm or greater, and having a surface with a hydrophobic vapor-transmissive moisture barrier integral therewith, the material or structure including one or more spunbonded, meltblown, coformed, bonded carded, or foamed constituents, optionally in combination with natural fibers, synthetic fibers or a mixture thereof.

### BRIEF DESCRIPTION OF THE FIGURES

FIG. 1 is a schematic representation of a conventional absorbent product having a topsheet and a non-permeable backsheet.

FIG. 1a is a schematic representation of a pore.

FIG. 2 is a schematic representation of a conventional absorbent product having a topsheet and a microporous backsheet with an apertured film layer.

FIG. 3 is a schematic representation of one embodiment of the present invention, including an optional microporous backsheet.

FIG. 4 is a photomicrograph generated by scanning electron microscopy (SEM) at a magnification of 80× of an untreated lower surface of an absorbent layer of a unitary absorbent core.

FIG. 5 is a photomicrograph generated by scanning electron microscopy (SEM) at a magnification of 80× of an treated lower surface of an absorbent layer of a unitary absorbent core.

FIG. 6 is a photomicrograph generated by scanning electron microscopy (SEM) at a magnification of 350× of an untreated lower surface of an absorbent layer of a unitary absorbent core.

FIG. 7 is a photomicrograph generated by scanning electron microscopy (SEM) at a magnification of 350× of an treated lower surface of an absorbent layer of a unitary absorbent core.

FIG. 8 is a photomicrograph generated by scanning electron microscopy (SEM) at magnifications of 45× and 80× of an untreated lower surface of an absorbent layer of a unitary absorbent core.

FIG. 9 is a photomicrograph generated by scanning electron microscopy (SEM) at magnifications of 250× and 450× of an untreated lower surface of an absorbent layer of a unitary absorbent core.

FIG. 10 is a photomicrograph generated by scanning electron microscopy (SEM) at magnifications of 45× and 80× of a treated lower surface of an absorbent layer of a unitary absorbent core with reticulated remnant of the barrier material emulsion.

FIG. 11 is a photomicrograph generated by scanning electron microscopy (SEM) at magnifications of 250× and 450× of a treated lower surface of an absorbent layer of a unitary absorbent core with reticulated remnant of the barrier material emulsion.

FIGS. 12(a) and 12(b) are photomicrographs at a magnifications of 7.5× and 40×, respectively of the unitary absorbent core of Example 25.

FIGS. 13(a) and 13(b) are photomicrographs generated by scanning electron microscopy (SEM) at magnifications of 35× and 100×, respectively, of the surface of the unitary absorbent core of Example 25.

FIGS. 14(a) and 14(b) are photomicrographs generated by scanning electron microscopy (SEM) at magnifications of 35× and 100×, respectively, of the cross-section of the unitary absorbent core of Example 25.

### DETAILED DESCRIPTION OF THE INVENTION

All U.S. patents cited herein are hereby incorporated by reference. In the case of a conflict in terminology, the present disclosure controls.

The unitary absorbent core of the present invention includes a fibrous, absorbent layer having an upper fluid receiving surface and a lower surface, and a vapor-transmissive moisture barrier integral with the lower surface of the absorbent layer.

The fibrous absorbent core may be formed using materials and techniques well known in the art. For example, the core may include one or more layers or strata of natural or synthetic fibers, hereinafter referred to as an “absorbent layer.” Cellulosic fibers are preferred for use in the absorbent layer. The absorbent layer may be formed using wetlaid or airlaid techniques, although airlaid processes are preferred. Binders, such as, for example, wet strength agents, latex emulsions, thermoplastic bicomponent fibers (“bico”) and combinations thereof, may be incorporated into the absorbent layer. The term “multibonded” is used to describe an absorbent layer incorporating a combination of binders including a preferred combination of latex and bico. Small amounts of a water-based hydrophilic emulsion binder may be applied to the surfaces of the absorbent layer to reduce “dust-off” of loose fibers and other particles. Further, for improved absorption of fluids, superabsorbent polymers (SAP) may be incorporated into the absorbent layer. SAP may be incorporated into the absorbent layer as particles, granules, flakes, etc., and may be included as a discrete stratum or mixed with the fibers of the absorbent layer. Materials such as fillers, perfumes, surfactants, and additives may be included in the core. Desirable absorbent cores suitable for use in the practice of this invention and components suitable for use in the cores are described in WO 99/16961, WO 99/63922, WO 99/63923, WO 99/63925, WO 00/41882, WO 00/38607, all of which are hereby incorporated by reference.

In a preferred embodiment, the unitary absorbent core of this invention can be described as a multi-zone or multi-strata or multilayer absorbent structure, which has two or more distinct strata. As used herein, the terms “stratum” and “strata” refer to the layered regions which make up the unitary structure. The unitary structure is constructed by assembling the strata in a continuous manner in a series of unit operations which results in the production of the unitary absorbent core. The strata of the unitary structure is not an assembly or laminate of preformed layers or plies which are assembled on a converting line. Notwithstanding the previous statement, in an optional variation of a preferred embodiment related to the continuous airlaid process of this invention, a carrier tissue of low basis weight or a separate stratum may be used to facilitate the production of a fibrous absorbent layer having a plurality of strata. In one embodiment, a preferred unitary absorbent core of this invention has two or more strata, at least one of which is a fibrous absorbent layer having an upper fluid receiving surface and a lower surface, and a vapor-transmissive moisture barrier integral with the lower surface of the absorbent layer. In a preferred embodiment, the unitary absorbent core is produced in a continuous manner using airlaid technology, where an individual forming head provides material for a single stratum and constitutes one unit operation in the series. Other unit operations in the series include application of a froth or foam which produces the vapor-transmissive moisture barrier, and may include compression and calendering and drying operations. The moisture barrier may be applied at any stage of the manufacture of the unitary absorbent core, e.g. after all the strata have been formed, or after any one or more strata have been formed.

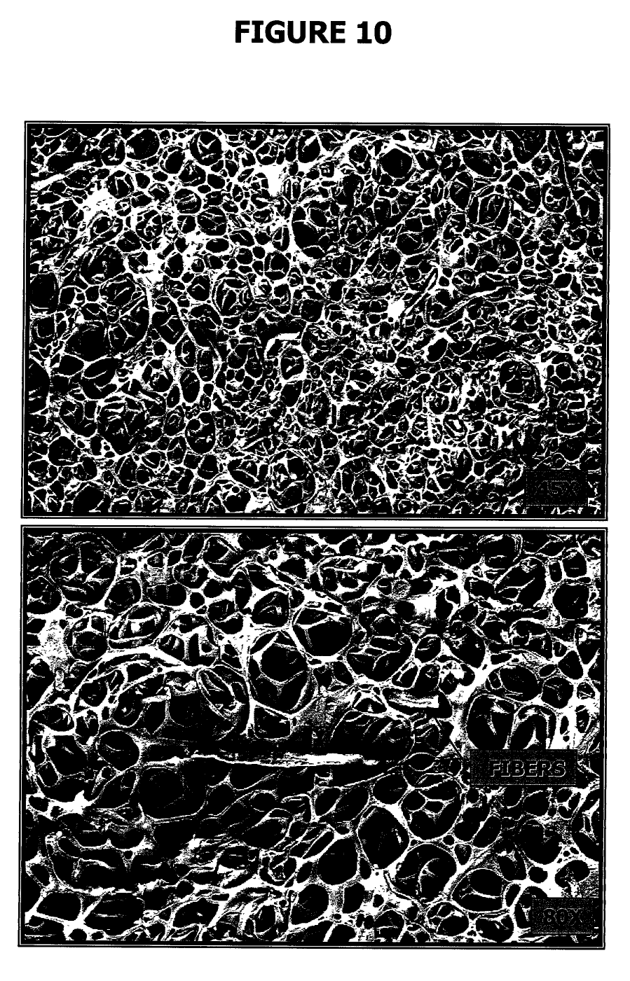

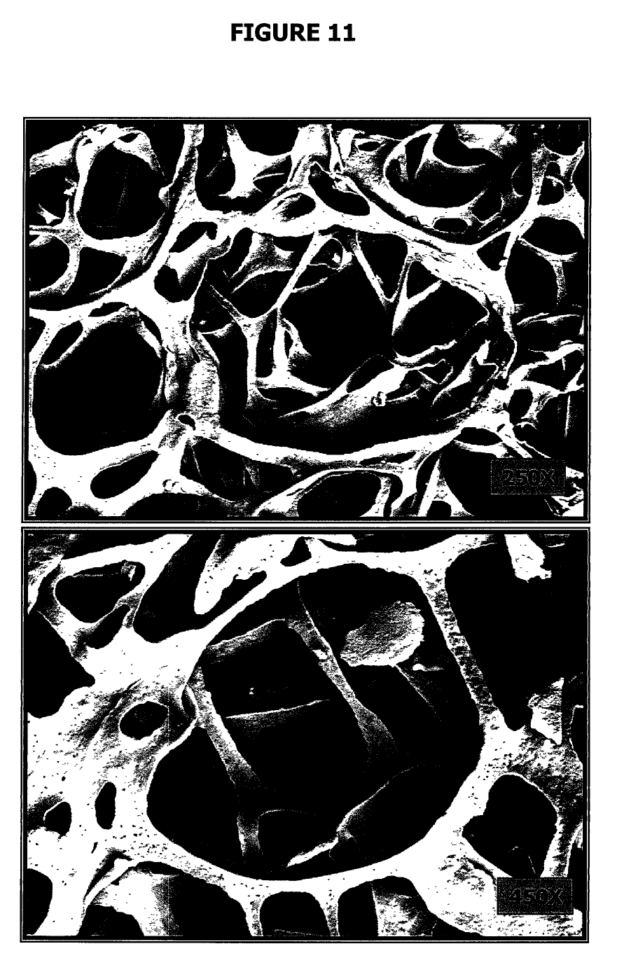

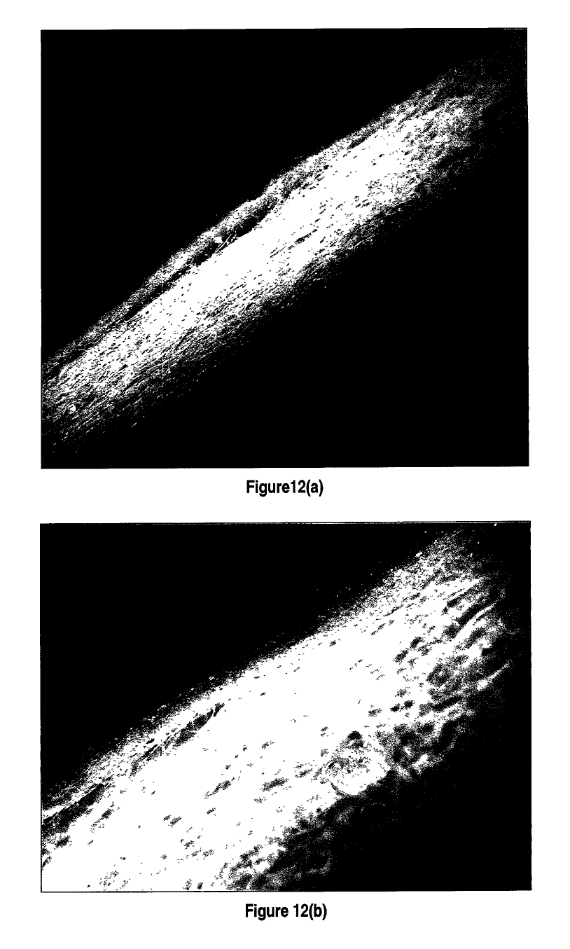

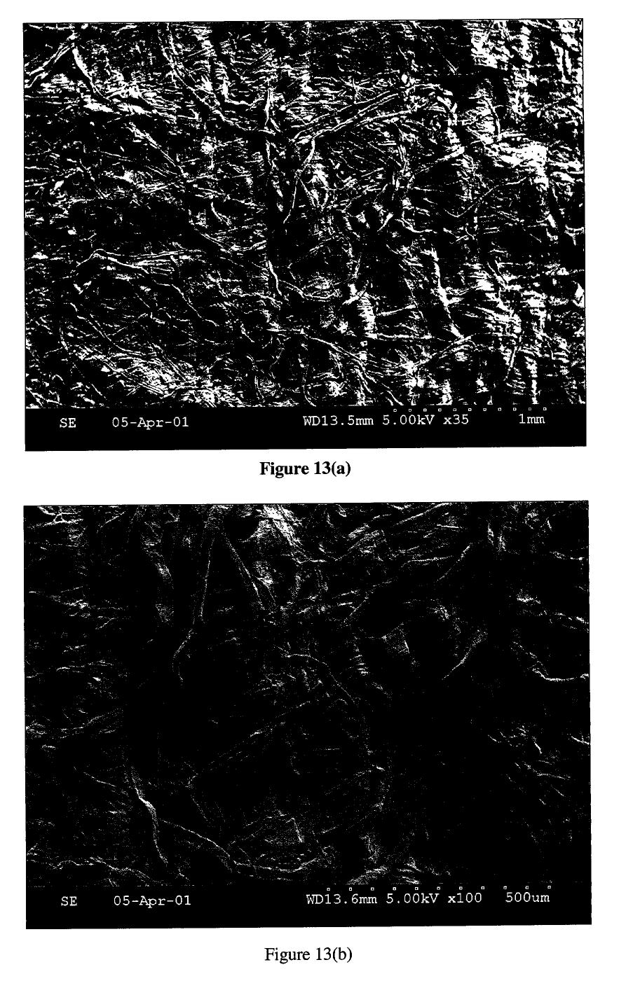

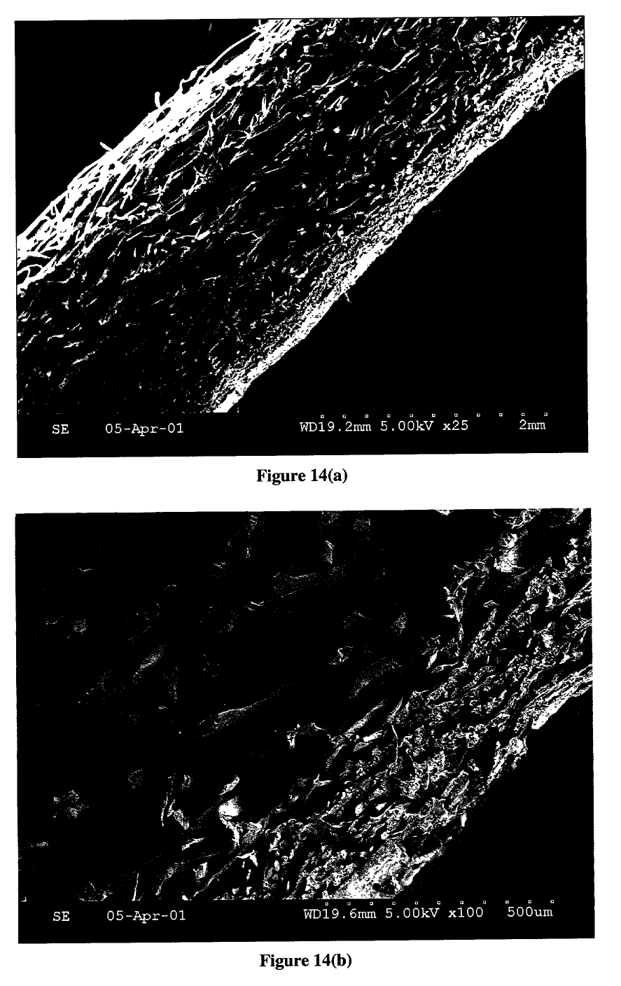

Generally herein, the term “froth” is used to describe foam that is of low viscosity and of poor stability, which is easily collapsible after application to the lower surface of the fibrous absorbent layer to form a hydrophobic vapor-transmissive moisture barrier integral with the lower surface of the absorbent layer wherein the moisture barrier has a structure which substantially includes fibers coated with hydrophobic material. The terms “stand-up foam” and “stand-up foam barrier” are used to describe a more substantial foam, which, after application to the lower surface of a fibrous absorbent layer to form a hydrophobic vapor-transmissive moisture barrier integral with the lower surface of the absorbent layer, results in some coating of fibers, but also wherein the moisture barrier has a reticulated remnant of a barrier material emulsion extending from the lower surface region of the absorbent layer to form an outer reticulated foam barrier. Moisture barriers with reticulated remnants of barrier material emulsions are shown in FIGS. 10 and 11.

The unitary absorbent core of this invention has a basis weight of about 75 gsm (grams per square meter) or greater, generally from about 80 to about 1000 gsm, and preferably from about 100 gsm to about 500 gsm, and more preferably from about 125 gsm to about 350 gsm.

In another embodiment, a breathable, partially fibrous or nonfibrous nonwoven material or structure including one or more spunbonded, meltblown, conformed, bonded carded, or foamed constituents has a basis weight of about 45 gsm or greater.

The unitary absorbent core of this invention has a density of from about 0.03 g/cc to about 0.7 g/cc, preferably from about 0.04 g/cc to about 0.3 g/cc.

The structures of this invention can include natural fibers, synthetic fibers or mixtures of both natural and synthetic fibers. Examples of the types of natural fibers which can be used in the present invention include fluffed cellulose fibers prepared from cotton, softwood and/or hardwood pulps, straw, keaf fibers, cellulose fibers modified by chemical, mechanical and/or thermal treatments, keratin fibers such as fibers obtained from feathers, bagasse, hemp, and flax, as well as man-made staple fibers made with natural polymers such as cellulose, chitin, and keratin. Cellulosic fibers include chemically modified cellulose such as chemically stiffened cellulosic fibers by crosslinking agents, fibers treated with mercerizing agents and cellulose acetate. Examples of suitable synthetic matrix fibers include polyethylene, polypropylene, polyester, including polyester terephthalate (PET), polyamide, polyacetates, cellulose acetate and rayon fibers. Certain hydrophobic synthetic fibers, such as polyolefins, may be surface treated with surfactant to improve wettability, or may be used untreated, depending upon their intended function within the core.

Examples of binders which may be useful in the absorbent structure of the present invention include polymeric binders in a solid or liquid form. The term “polymeric binder” refers to any compound capable of creating interfiber bonds between matrix fibers to increase the integrity of the stratum. At the same time, the binder may optionally bind fibers and SAP particles to each other.

For example, a dispersion of natural or synthetic elastomeric latex may be used as a binder. Thermoplastic fibers or powder, which are well known in the art, are also commonly used to provide bonding upon heating of the absorbent structure to the melting point of the thermoplastic fiber or powder. Other binders, which can be used for stabilizing the absorbent structure of the present invention, include bonding agents used to bond cellulose fibers. These agents include polymers dispersed in water, which are cured after application to the fibrous web and create bonds between fibers or between fibers and SAP particles. Examples of such agents include various cationic starch derivatives and synthetic cationic polymers containing crosslinkable functional groups such as polyamide-polyamine epichlorohydrin adducts, cationic starch, dialdehyde starch and the like. Any combination of the above-described polymeric binders may be used for stabilizing the structure of the present invention.

Binders useful in the structures of the invention include binders in liquid form or having a liquid carrier, including latex binders. Useful latex binders include vinyl acetate and acrylic ester copolymers, ethylene vinyl acetate copolymers, styrene butadiene carboxylate copolymers, and polyacrylonitriles, and sold, for example, under the trade names of Airbond, Airflex and Vinac of Air Products, Inc., Hycar and Geon of Goodrich Chemical Co., and Fulatex of H. B. Fuller Company. Alternatively, the binder may be a non-latex binder, such as epichlorohydrin and the like.

For bonding the fibers specifically, and for structural integrity of the unitary absorbent core generally, water-based latex binders may be used. Alternatively, or in combination with a latex binder, thermoplastic binding material (fibers or powders) may be used for bonding upon heating to the melting point of the thermoplastic binding material. Suitable thermoplastic binding material includes thermoplastic fibers, such as bicomponent thermoplastic fibers (“bico”). Preferred thermoplastic binding fibers provide enhanced adhesion for a wide range of materials, including synthetic and natural fibers, particles, and synthetic and natural carrier sheets. An exemplary thermoplastic bico fiber is Celbond Type 255 Bico fiber from KoSa.

Other suitable thermoplastic fibers include polypropylenes, polyesters, nylons and other olefins, or modifications thereof. A preferred thermoplastic fiber is FiberVisions type AL-Adhesion-C Bicomponent Fiber, which contains a polypropylene core and an activated copolyolefin sheath. In certain embodiments, the binder in the invention is a binding fiber, which is present in the absorbent structure in an amount which is less than about 10 percent by weight of the weight of SAP particles. In other embodiments of the invention, the binder fibers are present in an amount which is less than about 7 percent by weight of the weight of the absorbent structure.

Functional particles for use in the absorbent cores of the invention include particles, flakes, powders, granules or the like which serve as absorbents, odor control agents, such as, for example, zeolites or calcium carbonates, fragrances, detergents, antimicrobial agents and the like. The particles may include any functional powder or other particle having a particle diameter up to 3,000 μ(microns). In some preferred embodiments, the functional particles used in the core include super absorbent polymer particles (“SAP”). In one desirable embodiment of this invention, the unitary absorbent core contains from about 5 to about 90 percent by weight of SAP, preferably from about 10 to about 80 percent by weight of SAP, more preferably from about 10 to about 50 percent by weight of SAP.

U.S. Pat. Nos. 5,147,343; 5,378,528; 5,795,439; 5,807,916; and 5,849,211, which describe various superabsorbent polymers and methods of manufacture, are hereby incorporated by reference. Examples of the types of SAP particles which may be used in this invention, include superabsorbent polymers in their particulate form such as irregular granules, spherical particles, staple fibers and other elongated particles. The term “superabsorbent polymer” or “SAP” refers to a normally water-soluble polymer, which has been cross-linked. There are known methods of making water-soluble polymers such as carboxylic polyelectrolytes to create hydrogel-forming materials, now commonly referred to as superabsorbents or SAPs, and it is well known to use such materials to enhance the absorbency of disposable absorbent articles. There are also known methods of crosslinking carboxylated polyelectrolytes to obtain superabsorbent polymers. SAP particles useful in the practice of this invention are commercially available from a number of manufacturers, including Dow Chemical (Midland, Mich.), Stockhausen (Greensboro, N.C.), and Chemdal (Arlington Heights, Ill.). One conventional granular superabsorbent polymer is based on poly(acrylic acid) which has been crosslinked during polymerization with any of a number of multi-functional co-monomer crosslinking agents, as is well known in the art. Examples of multifunctional crosslinking agents are set forth in U.S. Pat. Nos. 2,929,154; 3,224,986; 3,332,909; and 4,076,673, all of which are hereby incorporated by reference. Other water-soluble polyelectrolyte polymers are known to be useful for the preparation of superabsorbents by crosslinking, these polymers include carboxymethyl starch, carboxymethyl cellulose, chitosan salts, gelatin salts, etc. They are not, however, commonly used on a commercial scale to enhance absorbency of disposable absorbent articles, primarily due to lower absorbent efficiency or higher cost.

Superabsorbent particulate polymers are also described in detail in U.S. Pat. Nos. 4,102,340 and Re 32, 649, both of which are hereby incorporated by reference. Suitable SAPs yield high gel volumes or high gel strength as measured by the shear modulus of the hydrogel. Such preferred SAPs contain relatively low levels of polymeric materials that can be extracted by contact with synthetic urine (so-called “extractables”). SAPs are well known and are commercially available from several sources. One example is a starch graft polyacrylate hydrogel marketed under the name IM1000 (Hoechst-Celanese; Portsmouth, Va.). Other commercially available SAPs are marketed under the trademark SANWET (Sanyo Kasei Kogyo; Kabushiki, Japan), SUMIKA GEL (Sumitomo Kagaku Kabushiki; Haishi, Japan), FAVOR (Stockhausen; Garyville, La.) and the ASAP series (Chemdal; Aberdeen, Miss.). Most preferred for use with the present invention are polyacrylate-based SAPs. As used in the present invention, SAP particles of any size or shape suitable for use in an absorbent core may be employed.

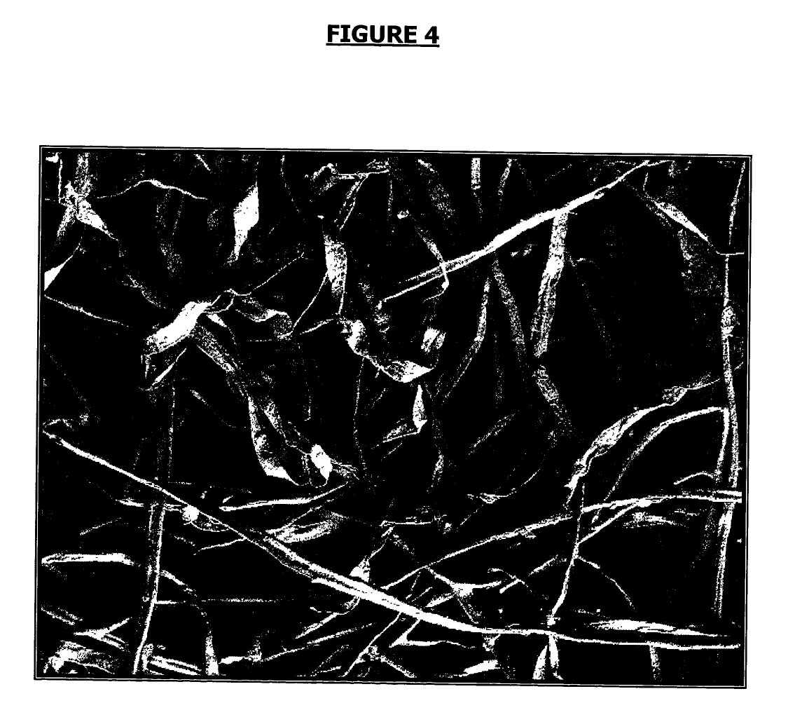

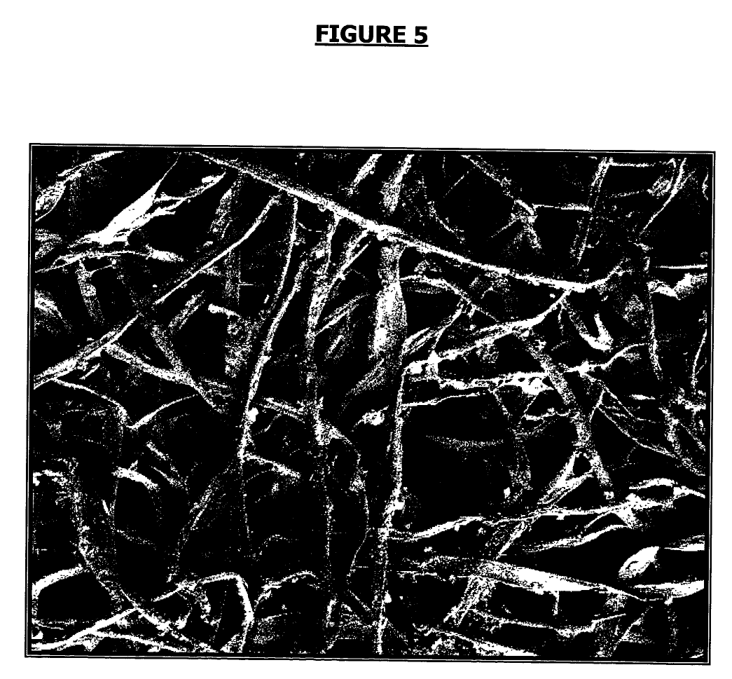

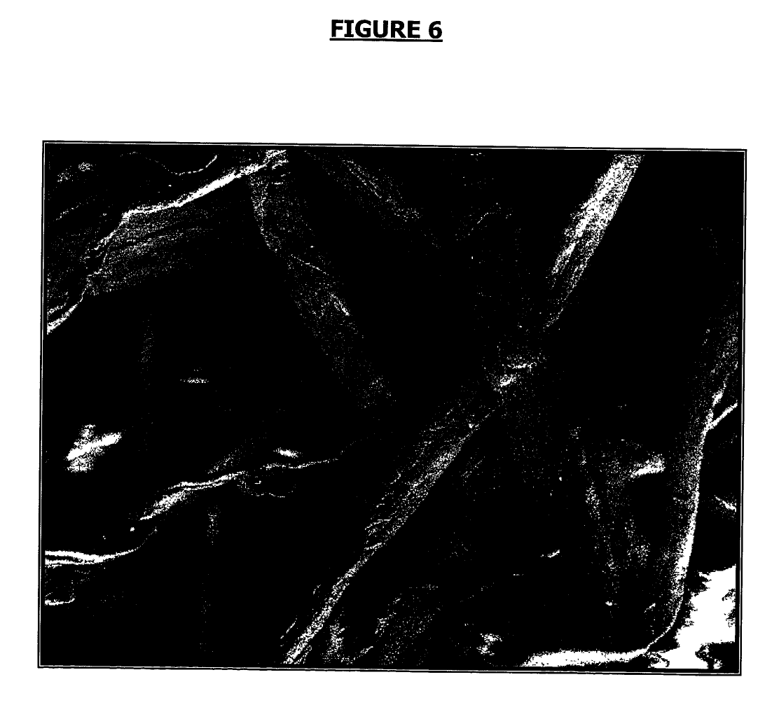

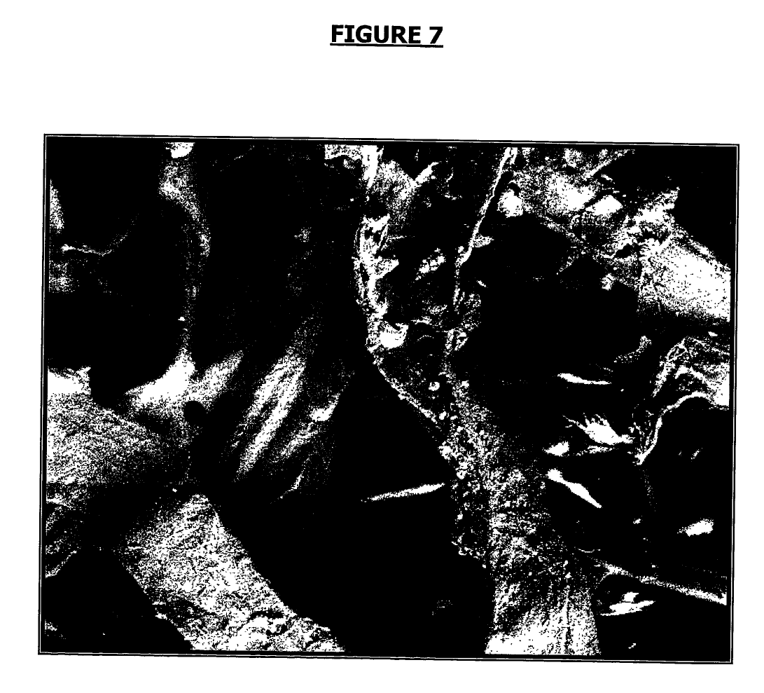

The vapor-transmissive moisture barrier integral with the lower surface of the absorbent layer is formed by applying a hydrophobic material to a fibrous substrate for which it is desirable to impart a barrier to the transmission of liquids, but for which it is also desirable to permit the passage of vapors including water vapor. The hydrophobic moisture barrier comprises a hydrophobic material which at least partially coats the fibers of the lower surface of the absorbent layer. The hydrophobic material can be a natural or synthetic polymer, or a mixture thereof. FIGS. 4 and 6 show the lower surface of the absorbent layer of an airlaid nonwoven absorbent core, as prepared in Example A below, which is untreated. FIGS. 5 and 7 show the treated lower surface of the absorbent layer of an airlaid nonwoven absorbent core, as prepared in Example B below. The term “vapor-transmissive moisture barrier integral with the lower surface of an absorbent layer” as used herein means that the barrier material at least partially coats at least some of the individual fibers of the absorbent layer, as shown in FIGS. 5 and 7, but that a continuous film is not formed. The absorbent layer remains vapor-transmissive since the pore structure between the untreated fibers, shown in FIGS. 4 and 6, remains substantially open after treatment to form the barrier, as shown in FIGS. 5 and 7. With the moisture barrier in place on the substrate, the unitary absorbent core has a hydrohead of 30 mm or greater as measured by modified EDANA nonwoven repellency test 120.1-80, a strikethrough of 1.8 g or less as measured by the standard strikethrough test, an air permeability of 18 m³/min/m² (60 ft³ /min/ft²) or greater as measured by modified ASTM D 737-96, and a water vapor transmission rate (WVTR) of 500 g/m^{2/}24 hr or greater. In one embodiment, the unitary absorbent core has a hydrohead of 85 mm or greater, a strikethrough of 0.08 or less, and an air porosity of 235 CFM or greater.

Within the scope of this invention is a vapor-transmissive moisture barrier integral with the lower surface of an absorbent layer where the hydrophobic barrier material coats at least some of the individual fibers of the absorbent layer, and where a reticulated remnant of a barrier material emulsion extends from the surface region of the absorbent layer to form an outer reticulated foam barrier as shown in FIGS. 10 and 11. In FIG. 10, the SEM photomicrograph at 80× shows several fibers intermingled with the reticulated remnant of the barrier material emulsion.

Hydrophobic materials suitable for use in this invention include a wide variety of materials known for water repellency, such as, for example, water insoluble thermoplastic organic materials including hydrocarbons and naturally occurring resins from petroleum, asphalt and coal tar, organic silicon compounds including polyorganosiloxanes, polysiloxanes containing halogens, especially fluorine, halohydrocarbons, especially polymers containing chlorine and fluorine, and various polymers in the form of natural or synthetic emulsions. Emulsion polymers suitable for use in this invention include lattices containing polymers, copolymers, as well as mixtures and blends of polymers and copolymers, containing in polymerized form one or more monomers of vinyl acetate, vinyl chloride, vinyl alcohol, acrylics, acrylates, acrylonitrile, ethylene, propylene, styrene, butadiene, isoprene, and various halogenated counterparts thereof.

In a preferred embodiment, the vapor-transmissive moisture barrier is formed by applying a hydrophobic polymeric latex emulsion to the lower surface of the absorbent layer. In at least one embodiment, it is desirable that a barrier is produced which has a contact angle for water on the film cast from an emulsion of about 80° or greater, as measured by the contact angle test (described below). Suitable hydrophobic polymeric emulsions include emulsions of both natural and synthetic polymers, including synthetic latexes. Several manufacturers supply such latex emulsions including Rohm and Haas, B.F. Goodrich, Air Products Polymers and Unichem Inc. A preferred latex emulsion is Unibond 0930 (Unichem Inc., Greenville, S.C.) which is an acrylic polymer. The emulsion can be applied by a variety of methods known in the art, including spray, brush, doctor blade, roller, and foam. Foam application is preferred.

The preferred application process involves the injection of air into an emulsion to form bubbles and create a temporary foam, or froth. In this application process, the collapse of the froth and elimination of air bubbles during the process of drying and curing the emulsion occurs. Advantages of foam application are more uniform reagent distribution, ability to apply reagent at higher solids contents, and more control over reagent penetration into the substrate.

For the embodiment of this invention where the moisture barrier produced has a reticulated remnant of a barrier material emulsion extending from the lower surface region of the absorbent layer to form an outer reticulated foam barrier, it is preferable to use a foam that has greater stability than the easily collapsible foams used for moisture barrier formation where no outer reticulated foam barrier is produced.

For a description of suitable conventional foaming procedures and foam stabilizers and foaming agents, reference is made to Mage, E. W., “Latex Foam Rubber,” John Wiley and Sons, New York (1962) and Rogers, T. H, “Plastic Foams”, Paper, Reg. Tech. Conf., Palisades Sect., Soc. Plastics Engrs., New York, November, 1964. Most common are the alkali metal, ammonia, and amine soaps of saturated or unsaturated acids having, for example, from about 12 to about 22 carbon atoms. Examples of suitable soaps include tallow soaps and coconut oil soaps, preferably the volatile amine or ammonia soaps, so that the volatile portion is vaporized from the foam. Other useful foaming-foam-stabilizing agents include lauryl sulfate-lauryl alcohol, lauryl sulfate-lauric acid, sodium lauryl sulfate, and other commonly used foamed stabilizers or foaming agents.

A preferred emulsion for the formation of the moisture barrier produced with a reticulated remnant of a barrier material emulsion extending from the lower surface region of the absorbent layer to form an outer reticulated foam barrier is Unibond 0938 from Unichem, which is an acrylic copolymer dispersed in a water base. Application by foam is preferred for Unibond 0938.

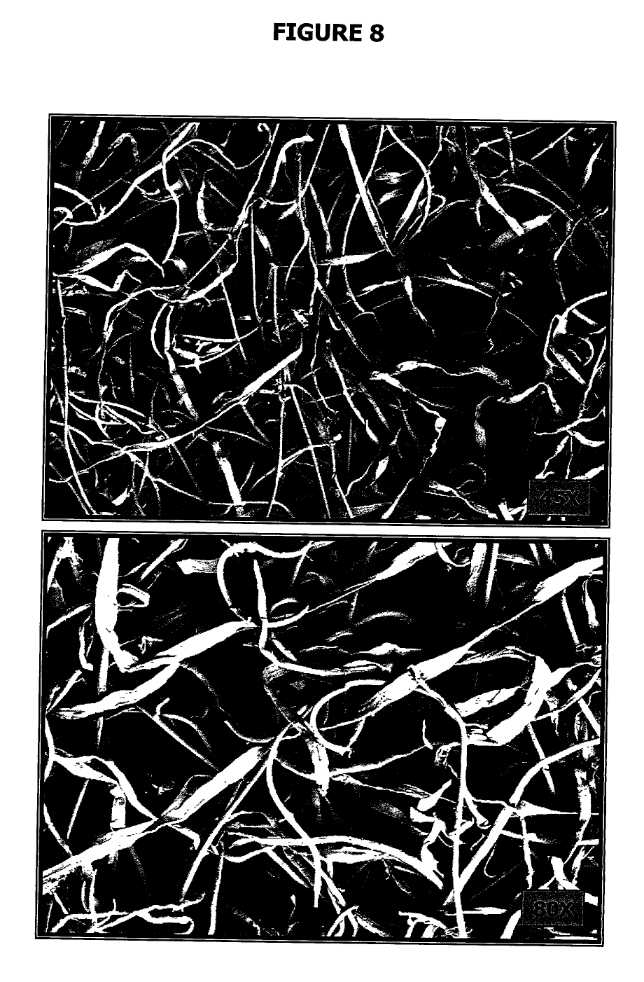

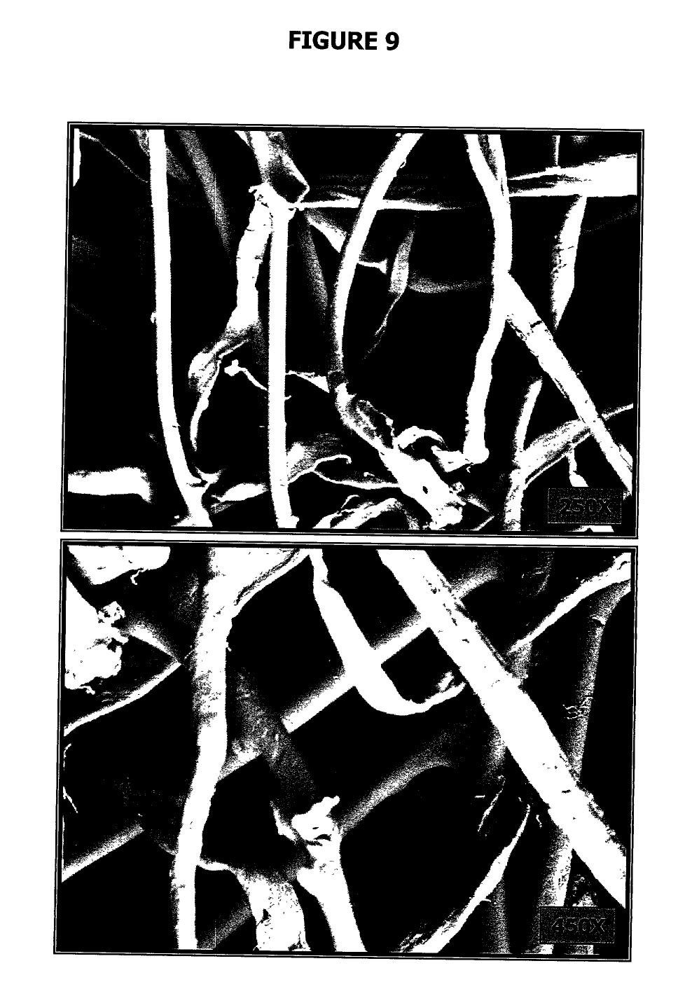

Unibond 0938 is engineered so that it does not collapse on the surface upon which it is foamed. After the Unibond 0938 foam is dried and cured, an elastic, reticulated structure, a reticulated remnant of the barrier material emulsion remains on the surface. See FIGS. 8-11, which are scanning electron micrographs (SEMs) of treated and untreated surfaces.

Generally, whether the moisture barrier formed has a reticulated remnant of the barrier material emulsion is a consequence primarily of the stability of the foam, which is influenced by the nature of the emulsion polymer in the emulsion, whether a foam stabilizer is used and the process conditions during application. In practice this is easily controlled.

After application of the latex emulsion to the surface of the absorbent layer, the emulsion is cured by removing water by drying or heat application. Optionally, crosslinking agents or other curing agents may be employed. Other additives may be included in the emulsion, such as biocides, water repellents, fillers and colorants.

Whichever application technique is used, it is important that the latex emulsion be applied in a sufficient quantity to at least partially coat a majority of individual fibers in the surface region of the absorbent layer. As used herein, “surface region” refers to the fibers of the absorbent layer directly exposed to the surface and several layers of fibers below such outermost fibers to a depth of from about 0.01 mm to about 1.0 mm from the surface, and preferably from about 0.05 mm to about 0.8 mm from the surface. As used herein, “partially coat” refers to the average portion of the surface area of a specific fiber coated with emulsion. Preferably, the fibers are coated by at least enough emulsion to render the fibers hydrophobic.

At the same time, it is important that the amount of latex emulsion applied not be so great that a continuous layer or film of polymer is formed which would block the pores. A continuous layer is disadvantageous because of the adverse affect on water vapor permeability of the resultant structure.

The amount of emulsion necessary to provide coated fibers without forming a continuous film or layer depends upon the density of the absorbent layer, the type of fibers employed, the type and physical properties of the emulsion employed, the method of application and the method of curing the absorbent core.

Without wishing to be bound by theory, it is believed that application of at least a partial coating of surface fibers with latex emulsion provides a hydrophobic moisture barrier, but because a continuous film or layer is not present, the pores created by adjacent coated fibers permit transmission of water vapor through the barrier.

In a preferred embodiment, the present invention includes a topsheet and an absorbent core treated with a hydrophobic latex emulsion as described herein. In a second preferred embodiment, a microporous backsheet may be included below the latex treated surface as shown in FIG. 3. A microporous material is available, for example, from Tredegar Film Products (Richmond, Va.) under the EXAIRE™ trade name. This material is a calcium carbonate-filled polyolefin film where pores are formed at the calcium/polymer interface sites when the film is deliberately stretched during production.

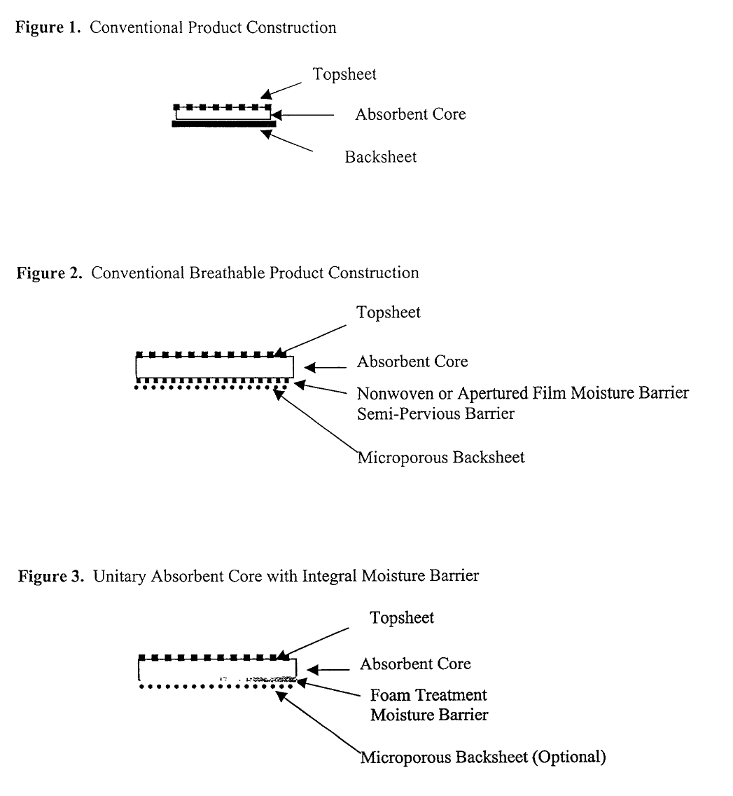

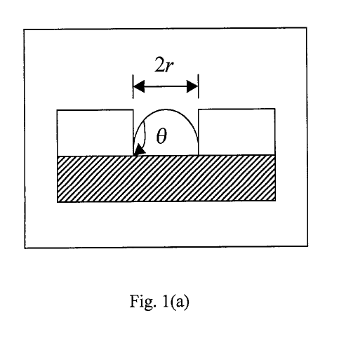

Fabric water repellency and breathability have been studied for several decades (A. W. Adamson, Physical Chemistry of Surfaces, Second Edition, Wiley, 1967, Chapters VII and X). A nonwoven web of fibers can be modeled as a bundle of cylindrical pores (capillaries) of radius r. See FIG. 1a. The fluid pressure required to penetrate the interfiber pores of a nonwoven web can be approximated from Laplace's equation for the penetration of a fluid into a tube:

P=(2γcosθ)/r

where:

P=pressure required to push fluid through the tube

γ=fluid surface tension

θ=advancing contact angle

r =pore radius

This equation can be used to describe web wetting (θ<90°, P is positive) or web water repellency (θ>90°, P is negative). In the case of water repellency, the fluid will not wet the web unless a pressure of P is applied to push the fluid into the web.

From the equation, barrier quality is predicted to be enhanced by increasing the contact angle with a water-repellent finish. In other words, the pores of the web should be rendered as hydrophobic as possible.

Apparent contact angles can be increased by surface roughness on the macroscale and microscale. Application of a waterproofing agent that causes microscopic pore surface roughness will lead to an increase in apparent contact angle, thus improving barrier quality.

From the equation, barrier quality is predicted to be enhanced by reducing the size of the interfiber pores. Ideally, the web should be as strong as possible. As pressure builds, weakness in the web will cause deformation, and deformation increases r, thus lowering pressure P. Web strength can be enhanced by, for example, increasing the amount of binder in the web.

The size of interfiber pores in a fibrous web is determined by the fiber size and the density or extent of compaction of the web. Increasing the density of the web can reduce the size of interfiber pores, or using smaller diameter fibers at the same density can reduce them. Smaller fibers pack together more efficiently in a densified web, resulting in smaller interfiber pores.

From the equation, using smaller fibers serves to decrease r, thus raising pressure P.

Filler material can be added to the hydrophobic emulsion to reduce the size of interfiber pores. From the equation, the addition of filler serves to decrease r, thus raising pressure P. The addition of filler to the treatment of the present invention increases barrier performance by partially blocking the pores of the nonwoven web, resulting in improved barrier quality. Filler suitable for use in the practice of this invention include calcium carbonate, various kinds of clay (bentonite and kaolin), silica, alumina, barium sulfate, sodium carbonate, talc, magnesium sulfate, titanium dioxide, zeolites, aluminum sulfate, cellulose-type powders, diatomaceous earth, magnesium sulfate, magnesium carbonate, barium carbonate, mica, carbon, calcium oxide, magnesium oxide, aluminum hydroxide, pulp powder, wood powder, cellulose derivative, polymer particles, chitin and chitin derivatives.

From the equation, barrier quality is predicted to be directly proportional to the fluid surface tension. The barrier treatment should be as durable as possible. Any additives in the barrier treatment that will dissolve in the fluid will likely lower its surface tension, thus lowering pressure P.

The contact angle test may be used to determine the contact angle of water on films cast from materials used to make the barrier, and in particular, water-based latex emulsions.

The emulsion is diluted with water to form a solution containing 10% solids. The solution is poured onto a borosilicate microscope slide to form a visible coat. The coated slide is set aside to dry overnight at ambient temperature and humidity. The coated slide is cured in a forced-air oven at 140° C. for five minutes. The advancing contact angle is measured using an FTÅ 200 Dynamic Contact Angle and Surface Tension Analyzer (First Ten Angstroms, Portsmouth, Va.) with reverse-osmosis treated water injected with a 27-gauge needle. The FTÅ 200 measures the advancing contact angle by the drop shape method.

Contact angles were measured for a naked slide (a “blank”), for Unibond 0930 and Unibond 0938 (both acrylic latex emulsions from Unichem Inc., Greenville, S.C.) and for Airflex 192 (ethylene-vinyl acetate latex emulsion, Air Products Polymers, Allentown, Pa.).

Water prefers to wet some surfaces and prefers to bead on others. A surface can be classified as hydrophilic, with a water contact angle less than 90°, or hydrophobic, with a water contact angle greater than 90°, based on the shape that a drop of water assumes when placed on that surface.

TABLE 1
Contact angle measurements for films cast from latex emulsions

| Material | Contact angle |
|---|---|
| Naked glass slide (blank) | 47.5 |
| Unibond 0930 | 95.9 |
| Unibond 0938 | 105.8 |
| Airflex 192 | 44.4 |

Table 1 shows results from contact angle measurements for films cast with Unibond 0930 and Unibond 0938 (Unichem Inc., Greenville, S.C.) and Airflex 192 (Air Products Polymers, Allentown, Pa.) latex emulsions. Table B-1 shows that Unibond 0930 and Unibond 0938 were both successful in rendering the surface of the microscope slide hydrophobic with a contact angle greater than 90°. Table B-1 shows that Airflex 192 was not successful in rendering the slide hydrophobic since it produced a contact angle less than 90°.

Any material capable of delivering a contact angle greater than 90° in this test would be a candidate for possible use in the present invention, provided that the material can be applied to a surface of an absorbent layer to render it hydrophobic without creating a continuous film which does not permit the passage of vapor. The hydrophobic emulsions Unibond 0930 and Unibond 0938 (Unichem Inc., Greenville, S.C.) are preferred latex emulsions for use in the practice of the present invention.

In an alternative process for the preparation of a unitary absorbent core comprising a fibrous absorbent layer having an upper fluid receiving surface and a lower surface with a hydrophobic vapor-transmissive moisture barrier integral with the lower surface of the absorbent layer, a hydrophobic material may be dissolved in a suitable solvent and contacted with the lower surface of the absorbent layer followed by causing the solvent to be removed. The solution may be applied to the lower surface of the absorbent layer by spraying, or the lower surface of the absorbent layer may be brought into contact with the solution by brief partial immersion, followed by draining and evaporation of the solvent.

In alternative embodiments of this invention, the fibrous absorbent layer of the absorbent core may be replaced wholly or in part by partially fibrous or nonfibrous structures capable of acceptable performance in an absorbent core, preferably a unitary absorbent core. Suitable partially fibrous or nonfibrous structures include spunbond webs, meltblown webs, coform webs, such as meltblown mixed with cellulose fibers, airlaid webs and bonded carded webs, differential basis weight nonwoven webs and high internal phase emulsion (HIPE) and other foam structures. In other embodiments, the hydrophobic vapor-transmissive moisture barrier of this invention may be integral with a surface of thermoset or thermoplastic cellular or noncellular material, which may be present in a composite of synthetic or synthetic and natural materials.

Breathable fibrous materials and unitary absorbent cores of this invention desirably have a hydrohead as measured by modified EDANA nonwoven repellency test 120.1-80 of 30 mm or more, preferably of 50 mm or more, more preferably of 70 mm or more, even more preferably of 90 mm or more, still more preferably of 200 mm or more.

Breathable fibrous materials and unitary absorbent cores of this invention desirably have a strikethrough as measured by the standard strikethrough test of 1.8 g or less, preferably of 1.2 g or less, more preferably of 0.7 g or less, even more preferably of 0.1 or less and still more preferably of 0.02 g or less.

Breathable fibrous materials and unitary absorbent cores of this invention desirably have an air permeability as measured by modified ASTM D 737-96 of 18 m³/min/m² (60 ft³/min/ft²) or greater, preferably of 31 m³/min/m² (100 ft³/min ft²) or greater, more preferably of 43 m³/min/m² (140 ft³/min/ft²) or greater, and even more preferably of 61 m³/min/m² (200 ft³/min/ft²) or greater.

Breathable fibrous materials and unitary absorbent cores of this invention desirably have water vapor transmission rate as measured by the water vapor transmission rate (WVTR) test which is a modification of ASTM E 96-95 of 500 g/m^{2/}24 hr or greater, preferably of 1000 g/m^{2/}24 hr or greater, more preferably of 2000 g/m^{2/}24 hr or greater, and even more preferably of 3000 g/m^{2/}24 hr or greater.

Breathable fibrous materials and unitary absorbent cores of this invention having a WVTR of 500 g/m^{2/}24 hr or greater desirably have barrier effectiveness values of 10 mm or greater, more desirably of 30 mm or greater, preferably of 50 mm or greater, more preferably of 75 mm or greater, still more preferably of 100 mm or greater and even more preferably of 230 mm or greater.

TEST METHODS

The following test methods were used to measure strikethrough, hydrostatic head and air porosity for the structures prepared in comparative example A and example B.

Frazier porosity—Air porosity of absorbent core samples was determined using an air permeability tester. Specifically, four handsheets per experimental sample were tested using the air permeability tester. For each handsheet, a pressure drop of 1.3 cm (one half inch) of water was established across the handsheet and air flow though the sheet was measured by the pressure drop across an orifice indicated on a vertical manometer. The average manometer reading was converted to air permeability using conversion tables.

Preparation of Synthetic Menses

The synthetic menstrual fluid used in these Examples contains the following ingredients in the designated amounts:

| | |
|---|---|
| Deionized water | 903.3 g |
| Sodium chloride | 9.0 g |
| Polyvinylpyrrolidone | 122.0 g |
| Biebrich Scarlet dye | 4.0 g |
| Total solution volume 1 liter | |

Biebrich Scarlet (red dye) can be obtained from Sigma Chemical Co., St. Louis, Mo. Polyvinylpyrrolidone (PVP, weight-average molecular weight approximately 55,000) can be obtained from Aldrich, Milwaukee, Wis. Sodium chloride (ACS grade) can be obtained from J. T. Baker, Phillipsburg, N.J. The dry ingredients are mixed in water for at least two hours to ensure complete dissolution. The solution temperature is adjusted to 22° C. exactly. Sixteen milliliters of solution is pipetted into the UL adapter chamber of a Brookfield Model DV-II+ viscometer (Brookfield Engineering Laboratories, Inc., Stoughton, Mass.). The UL spindle is placed into the chamber and the viscometer speed is set to 30 rpm. The target viscosity is between 9 and 10 centipoise. Viscosity can be adjusted with additional water or PVP.

Strikethrough Test for Moisture Barrier Samples are prepared into 10.3 cm×10.3 cm (4 in.×4 in.) squares. Each sample was placed onto a 10.3 cm×10.3 cm (4 in.×4 in.) Plexiglas backplate with the SAP-containing side facing up. The sample is covered with a 3.2 mm (0.125 in.) thick piece of 10.3 cm×10.3 cm (4in×4in) Plexiglass having a 3.2 cm (1.25 in) diameter hole in the center. A 5 ml insult of synthetic menses at room temperature is introduced through the opening. After the sample has been allowed to absorb the insult for 20 minutes, a tared stack of 10 Whatman #3 filter papers are placed beneath the prototype pad. A 2500g weight is placed on the plexiglass cover and allowed to stand for 2 minutes. After 2 minutes, the filter papers are removed and weighed. Strikethrough is calculated as follows:

Strikethrough (g)=Wet filter paper weight (g)−Tare filter paper weight (g)

Hydrostatic Head Test

Hydrostatic head is measured by employing a modified version of test method ISO 811:1981 - EN 20811:1992. The reported method is modified by employing a testing diameter of 60 mm; a cylinder length of 100 mm, a manometer diameter of 10 mm (internal), a dosing pump equipped with a T-valve for rapid cylinder filling, and employing a 10% w/v in water solution of calcium chloride (anhydrous, analytical reagent grade). The calcium chloride is employed to inhibit swelling of any SAP particles in the test sample, which might otherwise interfere with web integrity during the test.

### EXAMPLES

A 150 gsm multibonded airlaid nonwoven absorbent core containing 25% SAP was treated with hydrophobic latex material to form a moisture barrier on one surface of the web. The moisture barrier properties are measured as resistance to strikethrough under load and height of a column of water (hydrostatic head) required for strikethrough. Air permeability was measured as Frazier Air Porosity.

### COMPARATIVE EXAMPLE A

Untreated Web

A 150 gsm multibonded web was prepared. The web contained 69.7% fluff pulp (Foley fluff, Buckeye Technologies Inc., Memphis, Tenn., 12.0% bicomponent fibers (Type AL-Adhesion-C, Fiber Visions, Macon, Ga.; 1.3% Latex (Airflex 124 Vinyl Acetate-Ethylene Emulsion, Air Products and Chemicals, Allentown, Pa.); and 17.0% particulate polyacrylate superabsorbent (SXM 70, Stockhausen Inc., Greensboro, N.C.).

### EXAMPLE B

Web Treated With Hydrophobic Latex

One surface of the 150 gsm airlaid web described in Comparative Example 1 was coated with 10 gsm of Unibond 0930 latex (Unichem Corp, Greenville, S.C.). The coating process was based on foam coating. The hydrophobic latex was whipped into a free standing foam at 10% solids using a Kitchen Aid household blender and extruded onto the surface of the airlaid web. The foam was lightly calendered and the foam collapsed. The latex was then cured at 140 C. for 10 minutes.

TABLE 1a

| | Strikethrough | Hydrostatic Head | Air Porosity |
|---|---|---|---|
| Coating | (g) | (mm) | (CFM) |
| None | 2.25 | <5 | 211 |
| 10 gsm Unibond 0903 | 0.08 | 85 | 235 |

As can be seen from the data in Table 1a, the latex treated sample provided a greatly reduced strikethrough and a much higher hydrostatic head compared to the untreated control. At the same time, the permeability of the test structure was slightly better than the control.

The following test methods were used to measure water vapor transmission rate, air permeability, strikethrough and hydrostatic head for the structures prepared in the following examples.

Water vapor transmission rate

The method is used to determine the water vapor transmission rate (WVTR) through airlaid handsheets and is a modification of ASTM E 96-95.

Apparatus for this test includes a vapometer cup (#68-1, Thwing-Albert Instrument Co., Philadelphia, Pa.) and a forced-air oven capable of maintaining a temperature of 38° C. plus or minus 1° C. (Lindberg/Blue M, Lindberg/Blue M Co., Asheville, N.C., or equivalent). A circular sample 7.6 cm (three inches) in diameter is cut from a handsheet. One hundred milliliters of deionized water is placed into the vapometer cup. The test material is placed over the cup opening. The screw-on flange is tightened over the test material, leaving an exposed sample area of 33.17 square centimeters. The initial weight of the cup is recorded. The cup is placed on a tray and set in the forced-air oven for 24 hours at 38° C. After 24 hours, the cup is removed from the oven and reweighed to determine total water loss. WVTR is calculated as follows:

WVTR (g/m^{2/}24 hours)=[total water loss over 24 hours (g)×301.5]

The report for each test includes the average WVTR (n−3) for treated samples compared to the average WVTR (n=3) for the untreated control material. Note that the relative humidity within the oven is not specifically controlled in this test.

Air permeability

This method is a modification of the standard air permeability test for woven and nonwoven fabrics, ASTM D 737-96. Air permeability through the treated samples is compared with air permeability through untreated samples to give relative permeability effectiveness.

Air permeability of absorbent core handsheets is determined using an air permeability tester (Model 9025, modified with digital “A” and “B” gauges, U.S. Testing Co., Inc., 1415 Park Ave., Hoboken, N.J. 07030). Specifically, three handsheets per experimental sample (n=3) are tested using the air permeability tester. For each handsheet, a pressure drop of 1.3 cm (0.5 in.) of water is established across the handsheet. Airflow though the sheet is measured by the pressure drop across an orifice indicated on a vertical manometer. The average manometer reading is converted to air permeability using conversion tables provided by the manufacturer of the air permeability tester. Air permeability is reported as airflow in m³/min/m² and cubic feet per minute per square foot (ft³/min/ft²).

Strikethrough

This test is used to measure the resistance of sample materials to penetration by synthetic menses.

Samples are cut into 10.3 cm×10.3 cm (4 in.×4 in.) squares. Each sample is placed onto a 10.3 cm×10.3 cm (4 in.×4 in.) Plexiglas bottom plate with the treated side facing down. The sample is covered with a 3.2 mm (0.125 in.) thick, 10.3 cm×10.3 cm (4 in.×4 in.) Plexiglas top plate with a 3.2 cm (1.25-in.) diameter hole cut in its center. A 5 ml insult of synthetic menses (room temperature) is introduced through the hole in the top plate. After waiting for 20 minutes, a tared stack of 10 Whatman #3 filter papers, 110 mm circles, (Whatman International Ltd., England) is placed on the bottom plate beneath the sample. A 2500 g weight is placed on the Plexiglas top plate and is allowed to stand for 2 minutes. After 2 minutes, the filter papers are removed and weighed. Strikethrough is calculated as follows:

Strikethrough (g)=Wet filter paper weight (g)−Tare filter paper weight (g)

This test is usually run in triplicate (n=3) and the average value is reported in the unit of grams.

Hydrostatic head Hydrostatic head (hydrohead) is measured by using a modified version of the EDANA nonwoven repellency test 120.1-80. This EDANA test is based on test method ISO 811:1981 - EN 20811:1992. The EDANA method is modified by using a testing diameter of 60 mm; a cylinder length of 100 mm; a manometer diameter of 10 mm (internal); a dosing pump equipped with a T-valve for rapid cylinder filling; and an aqueous test solution of 10% (w/v) calcium chloride (General Chemical Co., Parsippany, N.J.). The calcium chloride is used to inhibit swelling of any SAP particles in the test sample, which might otherwise interfere with web integrity during the test. This test is usually run in triplicate (n=3) and the average result is reported in the unit of millimeters of hydrohead.

### EXAMPLES

The following examples are presented to provide a more detailed understanding of the invention. The specific materials and parameters are exemplary and are not intended to limit the scope of the invention.

### Examples 1 and 2

Laboratory Application of Frothed Emulsion

Example 1

Untreated Core

A three-layer, multibonded absorbent core was prepared on an airlaid pilot line containing three forming heads. The first or bottom layer of the core contained 40 gsm of fluff pulp (Foley Fluffs, Buckeye Technologies Inc., Memphis, Tenn.) and 5 gsm of bicomponent binder fiber (Type AL-Adhesion-C, 1.55 dpf×4 mm, FiberVisions, Macon, Ga). The second or middle layer contained 33 gsm of fluff pulp (Foley Fluffs, Buckeye Technologies Inc., Memphis, Tenn., and 7 gsm of bicomponent binder fiber (Type AL-Adhesion-C. 1.55 dpf×4 mm, FiberVisions, Macon, Ga.). The third or top layer contained 32 gsm of fluff pulp (Foley Fluffs, Buckeye Technologies Inc., Memphis, Tenn.), 6 gsm of bicomponent binder fiber (Type AL-Adhesion-C, 1.55 dpf×4 mm, FiberVisions, Macon, Ga.), 25 gsm of granular polyacrylate superabsorbent (Favor SXM 70, Stockhausen Inc., Greensboro, N.C.) and 2 gsm of latex adhesive (Airflex 124 ethylene-vinyl acetate emulsion, Air Products Polymers, Allentown, Pa.) sprayed on top for dust control. The absorbent core had an overall basis weight of 150 gsm and a density of 0.1 g/cc.

### Example 2

Laboratory Application of Hydrophobic Emulsion. The bottom surface (wire side) of the 150 gsm airlaid absorbent core described as Example 1 was coated with 9.0 gsm (dry basis) of Unibond 0930 latex emulsion (Unichem Corp., Greenville, S.C.). The core was treated in the laboratory using a process based on the application of a foam or froth. A water-based emulsion containing 10% latex solids and 1% frothing aid (Unifroth 0448, Unichem Inc., Greenville, S.C.) was whipped into froth using a household blender. The froth was placed onto the surface of the absorbent core with the aid of a screed. The froth was lightly calendered and the froth collapsed. The emulsion was dried and cured in a forced-air oven at 140° C. for 10 minutes.

TABLE 1
Test results for laboratory application of breathable barrier

| | | | | Air permeability, |
|---|---|---|---|---|
| | Barrier, | Hydrohead, | Strikethrough, | m³/min/m² |
| Example | gsm | mm | g | (ft³/min/ft²) |
| 1 | 0.0 | <5 | 2.25 | 64.3 (211) |
| 2 | 9.0 | 85 | 0.08 | 71.6 (235) |

The data in Table 1 shows that the treated core, Example 2, provided a reduced strikethrough and a higher hydrostatic head compared to the untreated “blank”, Example 1. At the same time, the air permeability of the treated core was slightly better than the control.

### Examples 3 through 7

Pilot-scale Application of Frothed Emulsion

### Example 3

Untreated Core

A three-layer, multibonded absorbent core was prepared on an airlaid pilot line containing three forming heads. The first or bottom layer of the core contained 40 gsm of Grade ND-416 pulp (Weyerhaeuser Co., Tacoma, Wash.) and 5 gsm of bicomponent binder fiber (Type AL-Adhesion-C, 1.55 dpf×4 mm, FiberVisions, Macon, Ga.). The second or middle layer contained 33 gsm of fluff pulp (Foley Fluffs, Buckeye Technologies Inc., Memphis, Tenn.), and 7 gsm of bicomponent binder fiber (Type AL-Adhesion-C, 1.55 dpf×4 mm, FiberVisions, Macon, Ga.). The third or top layer contained 32 gsm of fluff pulp (Foley Fluffs, Buckeye Technologies Inc., Memphis, Tenn.), 6 gsm of bicomponent binder fiber (Type AL-Adhesion-C, 1.55 dpf×4 mm, FiberVisions, Macon, Ga.), 25 gsm of granular polyacrylate superabsorbent (Favor SXM 70, Stockhausen Inc., Greensboro, N.C.) and 2 gsm of latex adhesive (Airflex 192 ethylene-vinyl acetate emulsion, Air Products Polymers, Allentown, Pa.) sprayed on top for dust control. The absorbent core had an overall basis weight of 150 gsm and a density of 0.1 g/cc.

### Example 4

Core Treated with Hydrophobic Emulsion on Pilot Line.

The bottom surface (wire side) of the 150 gsm airlaid absorbent core described as Example 3 was treated with 10 gsm (dry basis) of Unibond 0930 latex emulsion (Unichem Corp., Greenville, S.C.). The core was treated with the hydrophobic latex emulsion on an airlaid pilot line using a process based on the application of a foam or froth. A water-based emulsion containing 10% latex solids and a frothing aid (Unifroth 0448, Unichem Corp., Greenville, S.C., added to the emulsion in the amount of 0.5% based on total emulsion solids) was applied to the core as froth using a Gaston Systems applicator (Chemical Foam System, Gaston Systems Inc., Stanley, N.C.).

### Example 5

Additional Binder Fiber. The core was prepared as in Example 4, except that an additional 5 gsm of bicomponent binder fiber (Type AL-Adhesion-C, 1.55 dpf×4 mm, FiberVisions, Macon, Ga.) was added to the first or bottom layer of the absorbent core.

### Example 6

High Solids Application.

The core was prepared as in Example 4, except that the hydrophobic emulsion was applied to the core as a frothed, water-based emulsion composed of 20.8% latex solids and in the amount of 6.2 gsm (dry basis).

### Example 7

Increased Add On.

The core was prepared as in Example 6, except that the hydrophobic emulsion was applied in the amount of 10.4 gsm (dry basis).

TABLE 2
Test results for pilot line application of breathable barrier

| | | | Strike- | Air permeability, | |
|---|---|---|---|---|---|
| Ex- | Barrier, | Hydrohead, | through, | m³/min/m² | WVTR, |
| ample | gsm | mm | g | (ft³/min/ft²) | g/m²/24 hr |
| 3 | 0.0 | <5 | 2.72 | 47.8 (157) | 4192 |
| 4 | 10.0 | 35 | 1.79 | 46.0 (151) | n/d |
| 5 | 10.0 | 73 | 0.62 | 44.5 (146) | 4128 |
| 6 | 6.2 | 38 | 1.17 | 46.0 (151) | n/d |
| 7 | 10.4 | 60 | 0.11 | 43.3 (142) | 3800 |

Table 2 shows test results for Examples 3 through 7. Comparing the test results for Example 3 (untreated “blank”) with those for Example 4, Example 4 indicates that application of the barrier material, the hydrophobic emulsion, raises the hydrohead and, at the same time, lowers the amount of fluid that strikes through the core. Example 5 was prepared identically to Example 4, except that Example 5 contained twice the amount of bicomponent binder fiber in the bottom layer of the core compared to Example 4. Comparing the test results for Example 4 with those for Example 5, Table 2 shows that the additional binder fiber facilitates a boost in barrier properties by increasing hydrohead and decreasing strikethrough.

Example 7 was prepared identically to Example 6, except that an additional 4.2 gsm (dry basis) of hydrophobic emulsion was applied to Example 7. Table 2 shows that the additional emulsion serves to boost barrier properties by increasing hydrohead and decreasing strikethrough.

The micrographs of FIGS. 4 through 7 show that the pore size of the web does not appreciably change by the application of the barrier material. Table 2 corroborates the visual evidence of FIGS. 4 through 7, in that Table 2 shows that air permeability and WVTR do not change appreciably when the barrier is applied to the absorbent core.

### Examples 8 and 9

Addition of Fillers to the Hydrophobic Emulsion

### Example 8

Bentonite.

The following materials were combined to form a water-based emulsion with 10% latex solids and 3.3% bentonite clay: 75 g Unibond 0930 (Unichem Inc., Greenville, S.C., supplied as a water-based emulsion with 40% latex solids), 3 g Unifroth 0448 (Unichem Inc., Greenville, S.C.), 222 g water and 10 g bentonite clay (Black Hills Bentonite Co., Casper, Wyo.). The bottom surface (wire side) of the 150 gsm airlaid absorbent core described as Example 3 was treated with 9.4 gsm (dry basis) of the bentonite-containing emulsion. The core was treated in the laboratory using a process based on the application of a foam or froth. The bentonite-containing emulsion was whipped into froth using a household blender. The froth was placed onto the surface of the absorbent core with the aid of a screed. The froth was lightly calendered and the froth collapsed. The emulsion was dried and cured in a forced-air oven at 140° C. for 10 minutes.

### Example 9

Diatomaceous Earth.

The following materials were combined to form a water-based emulsion with 10% latex solids and 16.7% diatomaceous earth: 75 g Unibond 0930 (Unichem Inc., Greenville, S.C., supplied as an aqueous solution with 40% latex solids), 3 g Unifroth 0448 (Unichem Inc., Greenville, S.C.), 222 g water and 50 g diatomaceous earth (Celite Diatomite, Manville Products Co., Lompoc, Calif.). The bottom surface (wire side) of the 150 gsm airlaid absorbent core described as Example 3 was treated with 11.8 gsm (dry basis) of the diatomaceous earth-containing emulsion. The core was treated in the laboratory using a process based on froth application. The diatomaceous earth-containing emulsion was whipped into froth using a household blender. The froth was extruded onto the surface of the absorbent core. The froth was lightly calendered and the froth collapsed. The emulsion was dried and cured in a forced-air oven at 140° C. for 10 minutes. Note that Example 3 is the barrier substrate, or untreated “blank”, for Examples 4, 6, 7, 8 and9.

TABLE 3
Test results for laboratory application of breathable barrier.
Examples with fillers

| | | | Air permeability, |
|---|---|---|---|
| | Barrier, | Hydrohead, | m³/min/m² |
| Example | gsm | mm | (ft³/min/ft²) |
| 8, bentonite clay | 9.4 | 97 | 33.5 (110) |
| 9, diatomaceous earth | 11.8 | 69 | 35.4 (116) |

Table 3 shows hydrohead and air permeability data for Examples 8 and 9. Compared to Examples 4, 6 and 7, adding bentonite clay or diatomaceous earth to the hydrophobic emulsion serves to increase hydrohead at the expense of a modest drop in air permeability.

### Examples 10 and 11

A Lighter, Superabsorbent-free Core

### Example 10

Untreated Superabsorbent-free Core.

A two-layer, multibonded absorbent core was prepared on an airlaid pilot line using two forming heads. The first or bottom layer of the core contained 34.5 gsm of Grade ND-416 pulp (Weyerhaeuser Co., Tacoma, Wash.) and 5.5 gsm of bicomponent binder fiber (Type AL-Adhesion-C, 1.55 dpf×4 mm, FiberVisions, Macon, Ga.). The second or top layer contained 57.5 gsm of fluff pulp (Foley Fluffs, Buckeye Technologies Inc., Memphis, Tenn.), 9.5 gsm of bicomponent binder fiber (Type AL-Adhesion-C, 1.55 dpf×4 mm, FiberVisions, Macon, Ga.) and 3 gsm of latex adhesive (Airflex 124 ethylene-vinyl acetate emulsion, Air Products Polymers, Allentown, Pa.) sprayed on top for dust control. The core had an overall basis weight of 110 gsm and a density of 0.1 g/cc.

### Example 11

Treated Superabsorbent-free Core.

The bottom surface (wire side) of the 110 gsm airlaid absorbent core described as Example 10 was treated with 13 gsm (dry basis) of Unibond 0930 latex emulsion (Unichem Inc., Greenville, S.C.). The core was treated with the hydrophobic latex emulsion on an airlaid pilot line using a process based on the application of a foam or froth. A water-based emulsion containing 20% latex solids and a frothing aid (Unifroth 0448, Unichem Corp., Greenville, S.C., added to the emulsion in the amount of 0.5% based on total latex solids) was applied to the core as froth using a Gaston Systems applicator (Chemical Foam System, Gaston Systems Inc., Stanley, N.C.).

TABLE 4
Test results for pilot line application of breathable barrier.
Examples with superabsorbent-free absorbent core

| | | | | Air permeability, |
|---|---|---|---|---|
| | Barrier, | Hydrohead, | Strikethrough, | m³/min/m² |
| Example | gsm | mm | g | (ft³/min/ft²) |
| 10 | 0.0 | n/d | n/d | 42.1 (138) |
| 11 | 13.0 | 92 | 0.92 | 42.7 (140) |

Table 4 shows that application of the hydrophobic emulsion to the superabsorbent-free core resulted in a barrier with significant hydrohead without any loss of air permeability through the core.

### Examples 12 through 15

Additional Substrates

The substrates in Examples 12 through 15 were treated in the laboratory using a process based on the application of a foam or froth. A water-based emulsion containing 10% latex solids (Unibond 0930, Unichem Inc., Greenville, S.C.) and 1% frothing aid (Unifroth 0448, Unichem Inc., Greenville, S.C.) was whipped into froth using a household blender. The froth was placed onto the surface of the absorbent core with the aid of a screed. The froth was lightly calendered and the froth collapsed. The emulsion was dried and cured in a forced-air oven at 140° C. for 15 minutes.

### Example 12

Vizorb 3905

Vizorb 3905 is a commercial product of Buckeye Technologies Inc. (Memphis, Tenn.). Vizorb 3905 is formed on a tissue carrier, contains 24.5% granular polyacrylate superabsorbent, and has an overall basis weight of 250 gsm. Example 12 was treated with 2.3 gsm of hydrophobic emulsion (dry basis) on the tissue side of the substrate.

### Example 13

Vizorb 3004

Vizorb 3004 is a commercial product of Buckeye Technologies Inc. (Memphis, Tenn.). Vizorb 3004 is formed on a nonwoven carrier (spunbond polypropylene), contains no superabsorbent, and has an overall basis weight of 82 gsm. Example 13 was treated with 4.3 gsm of hydrophobic emulsion (dry basis) on the carrier side of the substrate.

### Example 14

Synthetic Nonwoven.

The substrate for Example 14 was a commercially available nonwoven (spunbond polypropylene), 22 gsm, obtained from Avgol Nonwoven Industries (Holon, Israel). Example 14 was treated with 10.1 gsm of hydrophobic emulsion (dry basis).

TABLE 5
Test results for laboratory application of
breathable barrier. Additional substrates

| | Basis weight, | Density, | Barrier, | Hydrohead, |
|---|---|---|---|---|
| Example | gsm | g/cc | gsm | mm |
| 12, Vizorb 3905 | 250 | 0.11 | 2.3 | 28 (0, untreated) |
| 13, Vizorb 3004 | 82 | 0.08 | 4.3 | 36 (0) |
| 14, synthetic | 22 | 0.10 | 10.1 | 39 (0) |
| nonwoven | | | | |

Table 5 shows hydrohead results for Examples 12 through 14. Table 5 shows that the breathable barrier of the present invention (as measured by hydrohead) can be built into a wide variety of substrates including airlaid, wetlaid and synthetic nonwovens. Examples 13 and 14 show that the barrier of the present invention can be formed on a synthetic nonwoven, and that the synthetic nonwoven can stand alone (Example 14) or it can be a component of a structure (Example 13). Example 12 shows that the barrier of the present invention can be formed on a wetlaid nonwoven (tissue).

### Example 15

Eucalyptus Fiber.

A two-layer thermal bonded absorbent core was prepared using a laboratory pad former (Buckeye design, Buckeye Technologies Inc., Memphis, Tenn.). The absorbent core contained 108 gsm of bleached eucalyptus kraft pulp (Aracruz Celulose USA, Raleigh, N.C.) and 12 gsm of bicomponent binder fiber (Type AL-Adhesion-C, 1.55 dpf×4 mm, FiberVisions, Macon, Ga.). The core had an overall basis weight of 120 gsm and a density of 0.10 g/cc. Example 15 was treated with 6.1 gsm of hydrophobic emulsion (dry basis).

TABLE 6
Test results for laboratory application of breathable barrier.
Eucalyptus absorbent core.

| | | | | | Air |
|---|---|---|---|---|---|
| | Basis | | | Strike- | permeability, |
| | weight, | Barrier, | Hydrohead, | through, | m³/min/m² |
| Example | gsm | gsm | mm | g | (ft³/min/ft²) |
| 15, eucalyptus | 120 | 6.1 | 140 | 0.00 | 21.9 (72) |

Typical fluff pulp used in absorbent cores (e.g. Foley Fluffs, Buckeye Technologies, Inc., Memphis, Tenn.; Grade ND-416, Weyerhaeuser Co., Tacoma, Wash.) is manufactured from coniferous wood, or softwood. It is well known to those skilled in the art that pulp fibers from deciduous wood, or hardwood, have a fiber length of about half and a fiber diameter of about half that of softwood pulp fibers. Table 6 shows hydrohead and strikethrough results for Example 15, constructed from eucalyptus hardwood pulp. Comparing all of the examples, the best hydrohead value and the lowest strikethrough value was obtained with Example 15.

### Examples 16 and 17

Laboratory and Pilot-scale Application of the Stand-up Foam Barrier

### Example 16

Laboratory Application of Hydrophobic Stand-up Foam

A three-layer, multibonded absorbent core was prepared on an airlaid pilot line containing three forming heads. The first or bottom layer of the core contained 16.3 gsm of fluff pulp (Foley Fluffs, Buckeye Technologies Inc., Memphis, Tenn.), 16.3 gsm of Grade HPF pulp (Buckeye Technologies Inc., Memphis, Tenn.), 8.0 gsm of bicomponent binder fiber (Type AL-Adhesion-C, 1.55 dpf×4 mm, FiberVisions, Macon, Ga.) and 1.5 gsm of latex adhesive (Airflex 192 ethylene-vinyl acetate emulsion, Air Products Polymers, Allentown, Pa.) foamed on the bottom for dust control. The second or middle layer contained 35.6 gsm of fluff pulp (Foley Fluffs, Buckeye Technologies Inc., Memphis, Tenn.), and 5.8 gsm of bicomponent binder fiber (Type 255, 2.8 dpf×4 mm, KoSa, Salisbury, N.C.). The third or top layer contained 33.1 gsm of fluff pulp (Foley Fluffs, Buckeye Technologies Inc., Memphis, Tenn.), 4.7 gsm of bicomponent binder fiber (Type 255, 2.8 dpf×4 mm, KoSa, Salisbury, N.C.), 26.3 gsm of granular polyacrylate superabsorbent (Grade 1186, Stockhausen Inc., Greensboro, N.C.) and 2.2 gsm of latex adhesive (Airflex 192 ethylene-vinyl acetate emulsion, Air Products Polymers, Allentown, Pa.) sprayed on top for dust control. The absorbent core had an overall basis weight of 150 gsm and a density of 0.1 g/cc. The bottom surface (wire side) of the 150 gsm airlaid absorbent core was treated with 48.8 gsm (dry basis) of Unibond 0938 latex emulsion (Unichem Corp., Greenville, S.C.). The core was treated in the laboratory using a process based on foam application. A water-based emulsion containing 50% latex solids and 1% frothing aid (Unifroth 0448, Unichem Inc., Greenville, S.C.) was whipped into foam using a household blender. The foam was placed onto the surface of the absorbent core with the aid of a screed. The emulsion was dried and cured in a forced-air oven at 140° C. for 15 minutes. Upon drying and curing, a reticulated polymeric structure, or stand-up foam, remained on the bottom surface of the core.

### Example 17

Pilot-line Application of Stand-up Foam

The bottom surface (wire side) of the base core of Example 16 was treated with 35.0 gsm (dry basis) of Unibond 0938 latex emulsion (Unichem Corp., Greenville, S.C.). The core was treated with the hydrophobic latex emulsion on an airlaid pilot line using a process based on foam application. A water-based emulsion containing 40% latex solids was applied to the core as foam using a Gaston Systems applicator (Chemical Foam System, Gaston Systems Inc., Stanley, N.C.). Upon drying and curing, a reticulated polymeric structure, or stand-up foam, remained on the bottom surface of the core.

TABLE 7
Test results for stand-up foam barrier, Examples 16 and 17

| | | | | Air permeability, |
|---|---|---|---|---|
| | Barrier, | Hydrohead, | Strikethrough, | m³/min/m² |
| Example | gsm | mm | g | (ft³/min/ft²) |
| 16 | 48.8 | 110 | 0.02 | 29.3 (96) |
| 17 | 35.0 | 111 | 0.00 | 44 (144) |

Table 7 shows test results for Examples 16 and 17 for the stand-up foam barrier. These examples provided minimal strikethrough and substantial hydrohead compared to the untreated cores of similar construction (Examples 1 and 3).

### Examples 18 and 19

Additional Pilot-scale Examples with Lower Barrier Basis Weight

### Example 18

Untreated Core

A two-layer, multibonded absorbent core was prepared on an airlaid pilot line using two forming heads. The first or bottom layer of the core contained 50 gsm of Grade ND-416 pulp (Weyerhaeuser Co., Tacoma, Wash.) and 7 gsm of bicomponent binder fiber (Type 255, 2.8 dpf×4 mm, KoSa, Salisbury, N.C.). The second or top layer contained 55 gsm of fluff pulp (Foley Fluffs, Buckeye Technologies Inc., Memphis, Tenn.), and 11 gsm of bicomponent binder fiber (Type 255, 2.8 dpf×4 mm, KoSa, Salisbury, N.C.), 25 gsm of granular polyacrylate superabsorbent (Favor SXM 70, Stockhausen Inc., Greensboro, N.C.) and 2 gsm of latex adhesive (Airflex 192 ethylene-vinyl acetate emulsion, Air Products Polymers, Allentown, Pa.) sprayed on top for dust control. The absorbent core had an overall basis weight of 150 gsm and a density of 0.1 g/cc.

### Example 19

Pilot-line Application of Stand-up Foam Barrier.

The bottom surface (wire side) of the base core described as Example 18 was treated with 20 gsm (dry basis) of Unibond 0938 latex emulsion (Unichem Corp., Greenville, S.C.). The core was treated with the hydrophobic latex emulsion on an airlaid pilot line using a process based on foam application. A water-based emulsion containing 41.8% latex solids was applied to the core as foam using a Gaston Systems applicator (Chemical Foam System, Gaston Systems Inc., Stanley, N.C.). Upon drying and curing, a reticulated polymeric structure, or stand-up foam, remained on the bottom surface of the core.

TABLE 8
Test results for stand-up foam barrier, Examples 18 and 19

| | | Hydro- | Strike- | Air permeability, | |
|---|---|---|---|---|---|
| | Barrier, | head, | through, | m³/min/m² | WVTR, |
| Example | gsm | mm | g | (ft³/min/ft²) | g/m²/24hr |
| 18 | 0.0 | <5 | 2.82 | 42.4 (139) | 4720 |
| 19 | 20.0 | 81 | 0.16 | 28 (92) | 4369 |

The data in Table 8 shows that the treated core, Example 19, provided a reduced strikethrough and a higher hydrostatic head compared to the untreated “blank”, Example 18. Concomitantly, the air permeability of the treated core was reduced 34% compared to the untreated core.

### Example 20

Laboratory Application of Stand-up Foam, Additional Substrate

Vizorb 3905 is a commercial product of Buckeye Technologies Inc. (Memphis, Tenn.). Vizorb 3905 is formed on a tissue carrier, contains 24.5% granular polyacrylate superabsorbent, and has an overall basis weight of 250 gsm. A water-based emulsion containing 40% latex solids (Unibond 0938, Unichem Inc., Greenville, S.C.) and 1% frothing aid (Unifroth 0448, Unichem Inc., Greenville, S.C.) was whipped into foam using a household blender. The foam was placed onto the tissue side of the Vizorb 3905 core with the aid of a screed. The emulsion was dried and cured in a forced-air oven at 140° C. for 15 minutes. Upon drying and curing, a reticulated polymeric structure, or stand-up foam, remained on the bottom surface of the core.

TABLE 9
Test results for laboratory application of
breathable barrier, additional substrate

| | Basis | Density, | Barrier, | Hydrohead, |
|---|---|---|---|---|
| Example | weight, gsm | g/cc | gsm | mm |
| 20, Vizorb | 250 | 0.11 | 24.3 | 160 |
| 3905 | | | | (0, untreated) |

Examples 21 and 22

Laboratory Application of Frothed Emulsion

### Example 21

Untreated Core.

A three-layer, multibonded absorbent core was prepared in the lab to simulate an airlaid pilot line containing three forming heads. The first or bottom layer of the core contained 18 gsm of grade 3024 tissue (CelluTissue, East Hartford, Conn.), 4.5 gsm of latex adhesive (Airflex 192 ethylene-vinyl acetate emulsion, Air Products Polymers, Allentown, Pa.) sprayed on bottom for holding tissue to pulp, 50 gsm of Grade Solucell 400 eucalyptus pulp (Klabin Bacell, Camacari BA Brasil). The second or middle layer contained 40 gsm of fluff pulp (Foley Fluffs, Buckeye Technologies Inc., Memphis, Tenn.), and 10 gsm of bicomponent binder fiber (Type AL-Adhesion-C, 1.55 dpf×4 mm, FiberVisions, Macon, Ga.). The third or top layer contained 15 gsm of bicomponent binder fiber (Type AL-Adhesion-C, 1.55 dpf×4 mm, FiberVisions, Macon, Ga.), and 3 gsm of latex adhesive (Airflex 192 ethylene-vinyl acetate emulsion, Air Products Polymers, Allentown, Pa.) sprayed on top for dust control. The absorbent core had an overall basis weight of 196.8 gsm and a density of 0.1 g/cc.

### Example 22

Laboratory Application of Hydrophobic Emulsion.

The bottom surface (wire side) of the 196.8 gsm airlaid absorbent core described as Example 21 was coated with 11.4 gsm (dry basis) of Unibond 0930 latex emulsion (Unichem Corp., Greenville, S.C.). The core was treated in the laboratory using a process based on froth application. A water-based emulsion containing 10% latex solids and 1% frothing aid (Unifroth 0448, Unichem Inc., Greenville, S.C.) was whipped into froth using a household blender. The froth was placed onto the surface of the absorbent core with the aid of a screed. The froth was lightly calendered and the froth collapsed.

The emulsion was dried and cured in a forced-air oven at 140° C. for 10 minutes.

TABLE 11
Test results for laboratory application of breathable barrier

| | Barrier, | Hydrohead, | WVTR, |
|---|---|---|---|
| Example | gsm | mm | g/m²/24hrs |
| 21 | 0.0 | <5 | N/d |
| 22 | 11.4 | 125 | 5106 |

The data in Table 11 shows that the combination of tissue and eucalyptus provides a breathable barrier with a significantly higher hydrostatic head and a high water vapor transmission rate.

### Examples 23-25

Pilot-scale Application of Frothed Emulsion

### Example 23

Untreated Core

A three-layer, multibonded absorbent core was prepared on an airlaid pilot line containing three forming heads. The first or bottom layer of the core contained 18 gsm of grade 3024 tissue (CelluTissue, East Hartford, Conn.), 2 gsm of latex adhesive (Airflex 192 ethylene-vinyl acetate emulsion, Air Products Polymers, Allentown, Pa.) sprayed on bottom for holding tissue to pulp, 40 gsm of Grade Solucell 400 eucalyptus pulp (Klabin Bacell, Camacari BA Brasil) and 20 gsm of bicomponent binder fiber (Type 255, 2.8 dpf×4 mm, KoSa, Salisbury, N.C.). The second or middle layer contained 40 gsm of fluff pulp (Foley Fluffs, Buckeye Technologies Inc., Memphis, Tenn.), and 20 gsm of bicomponent binder fiber (Type 255, 2.8 dpf×4 mm, KoSa, Salisbury, N.C.), and 30 gsm of granular polyacrylate superabsorbent (Favor SXM 70, Stockhausen Inc., Greensboro, N.C.). The third or top layer contained 40 gsm of fluff pulp (Foley Fluffs, Buckeye Technologies Inc., Memphis, Tenn.), 20 gsm of bicomponent binder fiber (Type 255, 2.8 dpf×4 mm, KoSa, Salisbury, N.C.), and 2 gsm of latex adhesive (Airflex 192 ethylene-vinyl acetate emulsion, Air Products Polymers, Allentown, Pa.) sprayed on top for dust control. The absorbent core had an overall basis weight of 220 gsm and a density of 0.07 g/cc.

### Example 24

Core Treated with Hydrophobic Emulsion on Pilot Line

The bottom surface (wire side) of the 220 gsm airlaid absorbent core described as Example 23 was treated with 20 gsm (dry basis) of Unibond 0930 latex emulsion (Unichem Corp., Greenville, S.C.). The core was treated with the hydrophobic latex emulsion on an airlaid pilot line using a process based on froth application. A water-based emulsion containing 20% latex solids and a frothing aid (Unifroth 1053, Unichem Corp., Greenville, S.C., added to the emulsion in the amount of 0.5% based on total emulsion solids) was applied to the core as froth using a Gaston Systems applicator (Chemical Foam System, Gaston Systems Inc., Stanley, N.C.).

### Example 25

Additional Latex Emulsion

A core was prepared as in Example 24, except that an additional 10 gsm of Unibond 0930 latex emulsion (Unichem Corp., Greenville, S.C.) was added to the first or bottom layer of the absorbent core.

TABLE 12
Test results for pilot line application of breathable barrier

| | Barrier | Hydrohead | Strikethrough | WVTR |
|---|---|---|---|---|
| Example | gsm | mm | G | g/m²/24hr |
| 23 | 0.0 | <5 | 2.85 | N/d |
| 24 | 20.0 | 203 | 0 | 3955 |
| 25 | 30.0 | 230 | 0 | 4534 |

The data in Table 12 shows that the combination of tissue and eucalyptus provides a breathable barrier with a significantly higher hydrostatic head and a high water vapor transmission rate.

### Example 26

Pilot-scale Application Forming Acquisition Layer, Absorbent Layer, Wicking Layer and Breathable Barrier Layer in a One Step Air Laid Process for a Unitary Absorbent Composite.

### Example 26

A Unitary Absorbent Composite

Acquisition layer, absorbent layer, wicking layer, and breathable barrier layer were prepared in a one step air laid pilot system. The first or bottom layer of the core contained 18 gsm of grade 3024 tissue (CelluTissue, East Hartford, Conn.), 45 gsm of Grade Solucell 400 eucalyptus pulp (Klabin Bacell, Camacari BA Brasil) and 5 gsm of bicomponent binder fiber (Type 255, 2.8 dpf×4 mm, KoSa, Salisbury, N.C.). The second or middle layer contained 50 gsm of chemically modified fluff pulp (HPF, Buckeye Technologies Inc., Memphis, Tenn.), and 9 gsm of bicomponent binder fiber (Type 255, 2.8 dpf×4 mm, KoSa, Salisbury, N.C.), and 50 gsm of granular polyacrylate superabsorbent (Favor 1180, Stockhausen Inc., Greensboro, N.C.). The third or top layer contained 35 gsm of PET fiber (Type 224, 15 denier×6 mm, KoSa, Salisbury, N.C.), and 6 gsm of latex adhesive (Airflex 192 ethylene-vinyl acetate emulsion, Air Products Polymers, Allentown, Pa.) sprayed on the top. The 10 gsm of breathable barrier layer (Unibond 0930 latex emulsion, Unichem Corp., Greenville, S.C.) was added to the bottom surface (wire side) of the airlaid absorbent composite. A water-based emulsion containing 20% latex solids and a frothing aid (Unifroth 0448, Unichem Corp., Greenville, S.C., added to the emulsion in the amount of 0.5% based on total emulsion solids) was applied to the composite as froth using a Gaston Systems applicator (Chemical Foam System, Gaston Systems Inc., Stanley, N.C.). The absorbent core had an overall basis weight of 228 gsm and a density of 0.13 g/cc.

TABLE 13
Test results for a breathable barrier composite

| | Barrier | Hydrohead | Strikethrough | WVTR |
|---|---|---|---|---|
| Example | gsm | mm | G | g/m²/24hr |
| 26 | 10.0 | 100 | 0.01 | 4134 |

The data in Table 13 is for Example 26, the pilot-scale production of a unitary abosrbent core with a fibrous absorbent layer with three strata produced in four separate unit operations to form an acquisition layer, absorbent layer, wicking layer, and hydrophobic vapor-transmissive moisture barrier integral with the lower surface of the absorbent layer in a continuous air laid process to produce a unitary absorbent with a high hydrostatic head and a high water vapor transmission rate.

Barrier Effectiveness Value

Hydrohead and strikethrough are two important attributes for a breathable moisture barrier. It is of interest to minimize strikethrough and, concomitantly, maximize hydrohead. A combination parameter, the barrier effectiveness value, can be devised with contributions from both hydrohead and strikethrough:

BEV=HH/(1+STV/HH₅₀)

where:

BEV=barrier effectiveness value, mm

HH=hydrohead, mm

STV=strikethrough, g

HH₅₀=strikethrough value chosen at which BEV equals 50% of the HH, g

In effect, the barrier effectiveness value penalizes the hydrohead for finite strikethrough. In this construction, the numerical value for hydrohead is reduced if strikethrough is finite. The higher the strikethrough, the more that hydrohead is reduced. In this construction, BEV equals HH when STV is zero. In addition, BEV equals half the HH when STV equals the HH₅₀. Any discussion of barrier effectiveness and BEV values assumes that the materials under consideration have a WVTR of 500 g/m²124 hr or greater.

TABLE 10
Barrier effectiveness values (BEV) for hydrohead and
strikethrough results presented in the examples

| | Hydrohead, | Strikethrough, | BEV (HH₅₀ = 0.75), |
|---|---|---|---|
| Example | HH, mm | STV, g | mm |
| 1 | 5 | 2.25 | 1.3 |
| 2 | 85 | 0.08 | 76.8 |
| 3 | 5 | 2.72 | 1.1 |
| 4 | 35 | 1.79 | 10.3 |
| 5 | 73 | 0.62 | 40.0 |
| 6 | 38 | 1.17 | 14.8 |
| 7 | 60 | 0.11 | 52.3 |
| 11 | 92 | 0.92 | 41.3 |
| 15 | 140 | 0.00 | 140.0 |
| 16 | 110 | 0.02 | 107.1 |
| 17 | 111 | 0.00 | 111.0 |
| 18 | 5 | 2.82 | 1.1 |
| 19 | 81 | 0.16 | 66.8 |

Table 10 shows barrier effectiveness values (BEV) for the examples for which both hydrohead and strikethrough were measured. Unitary absorbent cores of this invention desirably have a barrier effectiveness value of 30 mm or greater, more desirably of 50 mm or greater, and preferably of 75 mm or greater.

What is claimed is:

1. A unitary absorbent core having a basis weight of about 75 gsm or greater, comprising a fibrous absorbent layer having an upper fluid receiving surface and a lower surface with a hydrophobic vapor-transmissive moisture barrier integral with the lower surface of the absorbent layer.

2. The unitary absorbent core of claim 1, wherein the absorbent layer comprises natural fibers, synthetic fibers or a mixture thereof.

3. The unitary absorbent core of claim 1, wherein the hydrophobic moisture barrier comprises a hydrophobic material which at least partially coats the fibers of the lower surface of the absorbent layer.

4. The unitary absorbent core of claim 3 wherein the hydrophobic material is a natural or synthetic polymer.

5. The unitary absorbent core of claim 1 further comprising from about 5 to about 90 percent by weight of SAP.

6. The unitary absorbent core of claim 1, wherein the core has a basis weight of from about 80 gsm to about 1000 gsm.

7. The unitary absorbent core of claim 6, wherein the core has a basis weight of from about 100 gsm to about 500 gsm.

8. The unitary absorbent core of claim 1, wherein the core has a density of from about 0.03 to about 0.7 g/cc.

9. The unitary absorbent core of claim 8, wherein the core has a density of from about 0.04 to about 0.3 g/cc.

10. The unitary absorbent core of claim 1 having a hydrohead of 30 mm or more.

11. The unitary absorbent core of claim 10 having a hydrohead of 50 mm or more.

12. The unitary absorbent core of claim 11 having a hydrohead of 70mm or more.

13. The unitary absorbent core of claim 1 having a strikethrough of 1.8 g or less.

14. The unitary absorbent core of claim 13 having a strikethrough of 1.2 g or less.

15. The unitary absorbent core of claim 14 having a strikethrough of 0.7 g or less.

16. The unitary absorbent core of claim 1 having an air permeability of 18 m³/min/m² (60 ft³/min/ft²) or greater.

17. The unitary absorbent core of claim 1 having a water vapor transmission rate of 500 g/m²/24 hr or greater.

18. The unitary absorbent core of claim 17 having a water vapor transmission rate of 1000 g/m²/24 hr or greater.

19. The unitary absorbent core of claim 18 having a water vapor transmission rate of 2000 g/m²/24 hr or greater.

20. The unitary absorbent core of claim 19 having a water vapor transmission rate of 3000 g/m²/24 hr or greater.

21. The unitary absorbent core of claim 1 having a barrier effectiveness value of 30 mm or greater.

22. The unitary absorbent core of claim 21 having a barrier effectiveness value of 50 mm or greater.

23. The unitary absorbent core of claim 22 having a barrier effectiveness value of 75 mm or greater.

24. The unitary absorbent core of claim 1, wherein the moisture barrier has a structure which substantially is fibers coated with hydrophobic material.

25. The unitary absorbent core of claim 1, wherein the moisture barrier has a reticulated remnant of a barrier material emulsion extending from the lower surface region of the absorbent layer to form an outer reticulated foam barrier.

26. An absorbent article comprising:
(a) a liquid pervious top sheet, and
(b) a unitary absorbent core of claim 1.

27. The absorbent article of claim 22 further comprising a microporous backsheet.

28. The article of claim 26, wherein the article is an infant disposable diaper, a training pant, an absorbent surgical pad, an adult incontinence device, a sanitary napkin, a pantiliner or a feminine hygiene pad.

29. A process for the production of a unitary absorbent core having a basis weight of about 75 gsm or greater comprising a fibrous absorbent layer having an upper fluid receiving surface and a lower surface with a hydrophobic vapor-transmissive moisture barrier integral with the lower surface of the absorbent layer comprising:
(a) producing a fibrous absorbent layer having upper and lower surfaces,
(b) applying to the lower surface of the fibrous absorbent layer a hydrophobic material which at least partially coats at least some of the fibers of the lower surface of the absorbent layer.

30. The process of claim 29, wherein the fibrous absorbent layer comprises natural fibers, synthetic fibers or a mixture thereof.

31. The process of claim 29, wherein the hydrophobic material is a natural or synthetic polymer.

32. The process of claim 29, wherein the core comprises from about 5 to about 90 percent by weight of SAP.

33. The process of claim 29, wherein the hydrophobic material is an emulsion polymer.

34. The process of claim 23, wherein the emulsion polymer is applied in the form of a foam.

35. The process of claim 34, wherein the emulsion polymer includes a foam stabilizer.

36. Process of claim 34, wherein the emulsion polymer includes a hydrophobicity agent.

37. The process of claim 29, wherein the fibrous absorbent layer is a nonwoven produced by an airlaid process.

38. The process of claim 29, wherein the unitary absorbent core comprises two or more fibrous strata where each stratum is produced in a separate unit operation as part of a continuous process.

39. The process of claim 38, wherein the unitary absorbent core comprises three or more fibrous strata.

40. The process of claim 29, wherein the process comprises providing a tissue having a basis weight of less than about 30 gsm, spraying the tissues with emulsion polymer binder having a dry basis weight of about 10 gsm or less and airlaying a fibrous stratum thereupon.

41. The process of claim 40, wherein the fibrous stratum contains fifty percent or more by weight of eucalyptus fibers.

42. The process of claim 29, wherein the unitary absorbent core comprises one or more strata which are multibonded with an emulsion polymer binder and thermal bicomponent fiber binder.

43. The process of claim 29, wherein the moisture barrier produced has a structure which at least partially coats the fibers at the surface of the absorbent layer with hydrophobic material.

44. The process of claim 29, wherein the moisture barrier produced has a reticulated remnant of a barrier material emulsion extending from the lower surface region of the absorbent layer to form an outer reticulated foam barrier.

45. A unitary absorbent core produced by the process of claim 29.

46. A breathable nonwoven fibrous material having a basis weight of about 75 gsm or greater, a barrier effectiveness value of 30 mm or greater, and having a surface with a hydrophobic vapor-transmissive moisture barrier integral therewith comprising natural fibers, synthetic fibers or a mixture thereof, and a hydrophobic material which at least partially coats the fibers of a surface of the material.

47. A breathable, partially fibrous or nonfibrous nonwoven material or structure having a basis weight of about 45 gsm or greater, a barrier effectiveness value of 30 mm or greater, and having a surface with a hydrophobic vapor-transmissive moisture barrier integral therewith, the material or structure comprising one or more spunbonded, meltblown, coformed, bonded carded, or foamed constituents, optionally in combination with natural fibers, synthetic fibers or a mixture thereof.

48. The nonwoven material or structure of claim 47, wherein the foamed constituent is a high internal phase emulsion (HIPE) foam.

49. The nonwoven material or structure of claim 47, wherein the material or structure is a combination comprising from about 50 to about 99 percent by weight of natural fibers, synthetic fibers or a mixture thereof.

50. The nonwoven material or structure of claim 47, wherein the material or structure has been produced in a unitary process.
